(19) United States
(12) Patent Application Publication
(10) Pub. No.: US 2022/0118063 A1
(43) Pub. Date: Apr. 21, 2022

(54) METHODS AND COMPOSITIONS RELATED TO IMPROVED FACTOR VIII LONG HALF-LIFE COAGULATION COMPLEXES

(71) Applicant: CELL MACHINES, INC., Houston, TX (US)

(72) Inventors: James Kelly, Houston, TX (US); David G. Perryman, Decatur, GA (US)

(21) Appl. No.: 17/277,844

(22) PCT Filed: Sep. 19, 2019
(86) PCT No.: PCT/US2019/051881

§ 371 Date: Mar. 19, 2021

Related U.S. Application Data
(60) Provisional application No. 62/733,370, filed on Sep. 19, 2018.
(60) Provisional application No. 62/822,214, filed on Mar. 22, 2019.

(51) Int. Cl. A61K 38/48; A61K 47/54; A61K 47/60

(52) U.S. Cl. CPC A61K 38/4846; A61K 47/60; A61K 47/542

(57) ABSTRACT

Disclosed herein are methods and compositions related to improved coagulation factor complexes comprising factor VIII with a long half life. Disclosed herein is a coagulation factor complex comprising a factor VIII (FVIII) coagulation factor; a fusion protein comprising a D′D3 domain of von Willebrand's factor fused to full length albumin, or an albumin fragment; and an amino acid linker, in particular a cleavable amino acid linker.

### CROSS-REFERENCE TO RELATED APPLICATIONS

This application claims benefit of U.S. Provisional Application No. 62/733,370, filed Sep. 19, 2018, and to U.S. Provisional Application No. 62/822,214, filed Mar. 22, 2019, both of which are incorporated herein by reference in their entirety.

### BACKGROUND

Blood coagulation is controlled by a very complicated series of checks and balances such that coagulation only is triggered in the event of a bleed (Smith, Travers, & Morrissey, 2015). Injury sets off activation of these enzymes, resulting in an amplifying cascade of reactions that seals the wound. Hemophilia results from a defect in a gene coding for one of these proteins such that the cascade is aborted prematurely and bleeding continues. The most common forms of hemophilia, hemophilia A, hemophilia B and von Willebrand's disease, have long been treated by infusion of purified factor concentrates, replacing the defective enzyme and restoring the ability of the blood to clot.

Infusion of factor is remarkably effective, allowing afflicted individuals who may have died in childhood to have normal life expectancies (Hoots, 2003). With the increasing use of prophylaxis, that is, regularly scheduled infusions of factor to maintain a reasonable level of protection, these patients can lead essentially normal lives (Srivastava et al., 2012). This does not come without cost, literally and figuratively. Patients with severe hemophilia A need to infuse factor every other day due to the short circulatory half life of Factor VIII (FVIII), the protein missing in that form of the disease. This creates a number of problems, such as continued venous access and noncompliance.

Another very serious problem is encountered when patients develop inhibitory antibodies to the infused FVIII (Kempton & Meeks, 2014). About 30% of all hemophilia A patients will develop antibodies at some point in their therapy but about 5% develop such a serious inhibitor problem that FVIII infusion is no longer effective. This necessitates the use of “bypass” therapy. Factor VIIa (FVIIa) is one of the initiators of the coagulation cascade and can be used to step around the need for either FVIII or Factor IX (FIX) in the process. This requires very high concentrations of FVIIa and very frequent dosing since FVIIa has a circulatory half life of only two hours.

Because of these and other reasons, subcutaneously available, extended half-life factors (SCEHL) are very desirable (Pipe, 2010). Less frequent, subcutaneous dosing should improve compliance, solve venous access problems and expose the patient to a smaller mass of purified protein, perhaps reducing inhibitor formation. Moreover, SCEHL proteins could expand treatment to the estimated 70% of hemophilia patients worldwide who are still untreated. Cost of factor is major issue but so is the complicated medical service required for hemophilia patients, particularly children. Since factor needs to be infused intravenously, rather than simply being injected subcutaneously, children with severe disease are most frequently treated at specialized hemophilia treatment centers. An obvious impediment to their treatment is that they must be delivered to the center several times per week which, especially in less developed countries, can put therapy beyond reach. Factors that persisted for longer periods and that could be injected subcutaneously could reduce or even eliminate these trips.

This problem has been recognized for some time and there have been numerous attempts to prolong the half life of factors. There are two common strategies for increasing the half life of therapeutic proteins. The first is to modify the proteins with chains of polyethylene glycol, commonly called PEGylation (Ginn, Khalili, Lever, & Brocchini, 2014). The PEG chains increase the water of hydration around the protein resulting in reduced affinity for certain receptors and antibodies. The second strategy is to make use of the neonatal Fc receptor (FcRN) via fusion of the target protein with either the Fc portion of the immunoglobulins or human serum albumin (Andersen et al., 2011). Both immunoglobulins and albumin have long circulatory half lives due to their interaction with and protection by FcRN. When albumin or immunoglobulins are internalized in a variety of cells, they bind to FcRN and are recycled to the surface rather than being degraded. Both of these proteins have half lives of several weeks as a result.

These strategies have been successfully utilized to increase the half life of human Factor IX, the protein involved in hemophilia B (Mannucci & Mancuso, 2014). They have been less successful in prolonging the half life of FVIII (Buyue et al., 2014; Stennicke et al., 2013). FVIII itself is an unstable protein and requires the presence of von Willebrand Factor (vWF). FVIII in the absence of vWF has a half life of only a few minutes. The half life of the complex is determined by the half life of vWF so modifications to FVIII have only a small effect, increasing half-life from 12 hours to 18 hours.

Accordingly, there is a need for compositions and methods for long half-life coagulation complexes. Among other improvements, the present invention satisfies this need by providing the surprising discovery that a D′D3short domain of von Willebrand's coagulation factor is sufficient and advantageous for the creation of improved coagulation factor complexes with exceptional half-life.

### SUMMARY

Disclosed herein is a coagulation factor complex comprising a factor VIII (FVIII) coagulation factor; a fusion protein comprising a D′D3 domain of von Willebrand's factor fused to full length albumin, or an albumin fragment; and an amino acid linker, in particular a cleavable amino acid linker.

Among other improvements, the present invention provides the surprising discovery that a D′D3short domain of von Willebrand's coagulation factor is sufficient and advantageous for the creation of improved coagulation factor complexes with exceptional half-life. Thus, also disclosed is a coagulation factor complex comprising a factor VIII (FVIII) coagulation factor; a fusion protein comprising a D′D3short fragment of von Willebrand's factor fused to full length albumin, an albumin fragment, an immunoglobulin Fc domain, or an Fc fragment. In some aspects, the coagulation factor complex can further comprise an amino acid linker.

Also disclosed is a method of making a coagulation factor complex, the method comprising: introducing a D′D3 domain or the D′D3short of von Willebrand's factor to full-length human albumin, an albumin fragment or variant thereof, an immunoglobulin Fc domain, or an Fc fragment or variant thereof to form a fusion protein; introducing the fusion protein to coagulation factor FVIII (FVIII), wherein FVIII binds a receptor of D′D3 or D′D3short, thereby forming a coagulation factor complex.

Disclosed are kits comprising the coagulation factor complexes disclosed herein.

Also disclosed are methods of treating a subject with a disease requiring coagulation factor infusion, the method comprising administering to the subject the coagulation factor complex disclosed herein. The disease can be hemophilia, for example. The administration of the coagulation factor complex to the subject result can result in a blood level half-life of the coagulation factor complex which is greater than the blood level half-life obtained upon administration of the coagulation factor alone. The coagulation factor complex can be administered to the subject via injection (for example, subcutaneous injection), inhalation, internasally, or orally.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows a scheme for the construction of a long half-life coagulation factor VIII (coagulation factor complex, including a modified coagulation factor).

FIGS. 2A, 2B, 2C, and 2D shows the production and purification of CM110. FIG. 2A shows CM110 accumulates in the supernate of Expi293 cells. FIG. 2B shows CM110 is purified from the cell supernate using a human serum albumin affinity column FIG. 2C shows SDS gel analysis of fractions from purification. M-molecular weight markers, Crude—cell supernate, Flow—flow through from affinity column, Eluate—CM110 eluted from the affinity column with 2M MgCl2. FIG. 2D shows the protein passed over a Superdex S-200 gel filtration column.

FIGS. 3A, 3B, and 3C show labeling of CM110 and FVIII. FIG. 3A shows that CM110 was labeled with N-hydroxysuccinimide-PEG12-TCO, followed by reaction with fluoresceinyl tetrazine and run on an SDS polyacrylamide gel. Measuring fluorescence of the labeled compound against a standard curve yielded 10 molecules of NHS-P12-Tet per CM110. FIG. 3B shows that FVIII was labeled with fluoresceinyl maleimide and run on an SDS polyacrylamide gel. Comparison with a standard curve yielded 1.4 molecules per FVIII. FIG. 3C shows thrombin treatment of labeled FVIII. Domain A1 and A2 are both labeled.

FIGS. 4A and 4B show formation of CM211, the complex between a CM110 and a FVIII. FIG. 4A shows that CM211 emerges from a Superdex S-200 Increase column at a molecular weight of about 430,000 daltons. FIG. 4B show SDS gel electrophoresis of CM211.

FIGS. 5A and 5B show that high salt does not release free FVIII from CM211. FIG. 5A shows that CM211 was treated with 0.25 M CaCl2 then run on a Superdex S-200 Increase column in buffer containing 0.25 M CaCl2. All of the FVIII activity remains in the high molecular weight range. FIG. 5B shows that CM211 was treated with 0.8 M NaCl then run on a Superdex S-200 Increase column in buffer containing 0.8 M NaCl. All of the FVIII activity remains in the high molecular weight range.

FIGS. 6A and 6B. CM211 corrects the activated partial thromboplastin time in FVIII deficient plasma. FIG. 6A shows FVIII in FVIII-deficient plasma. FIG. 6B shows CM211 in FVIII-deficient plasma.

FIGS. 7A and 7B. CM211 in a thrombin generation test. FIG. 7A shows CM211 generates the same amount of thrombin as FVIII. FIG. 7B shows CM211 has the same area under the curve (AUC) as FVIII in a thrombin generation test. The FVIII control generated 209 nM thrombin with an area under the curve of 3,246.

FIGS. 8A, 8B, and 8C show mouse studies. FIG. 8A shows measurement of CM110 half life—92 hrs. FIG. 8B shows measurement of CM211 half life—55 hrs.

FIG. 8C shows test of subcutaneous availability of CM211.

FIGS. 9A, 9B, and 9C show the production and purification of a CM110short containing only the D′ fragment of vWF. FIG. 9A shows SDS gel electrophoresis of 1) molecular weight markers, 2) supernate from control Expi293 cells, 3) supernate from Expi293 cells transfected with pCM110shortD′ and 4) protein eluted from the albumin affinity column and probed with an anti-albumin antibody. FIG. 9B shows chromatography of protein eluted from affinity column on Superdex 200 Increase size exclusion column. FIG. 9C shows SDS gel electrophoresis of purified a CM110short containing only D′.

FIG. 10. Schematic for release of the various components of CM211 after treatment with thrombin.

FIG. 11. Cleavage of a CM110short containing only D′ with thrombin showing release of free albumin and D′.

FIG. 12. Demonstration that there is only a single free sulfhydryl in a CM110short containing only D′. Panel 12A. Measurement of incorporation of fluoresceinyl maleimide into a CM110short containing only D′ and calculation to demonstrate one fluorescein per this CM110short. Panel 12B. Treatment of fluorescently labeled CM110short containing only D′ with thrombin to demonstrate that the label stays with the albumin fragment.

FIG. 13. Scheme for the construction of a CM211s using click chemistry to incorporate a cleavable thrombin peptide into the chemical linker.

FIG. 14. Chromatography of a CM211s on Superdex 200 Increase demonstrating that all of the FVIII activity moves to a higher molecular weight with formation of the complex.

FIG. 15. SDS acrylamide gels of labeled and unlabeled CM115. Panel 15A. Gel stained for protein. Lane 1: Molecular weight markers, Lane 2: blank, Lane 3: CM115, Lane 4: CM115 treated with thrombin, Lane 6: human albumin Panels 15B. Same gel showing the fluorescent label Alexa488. Lane 1: Molecular weight markers, Lane 2: blank, Lane 3: CM115, Lane 4: CM115 treated with thrombin, Lane 6: human albumin.

### DETAILED DESCRIPTION

The materials, compositions, and methods described herein can be understood more readily by reference to the following detailed descriptions of specific aspects of the disclosed subject matter and the Examples and Figures included herein.

Before the present materials, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### Definitions

In this specification and in the claims that follow, reference will be made to a number of terms, which shall be defined to have the following meanings:

Throughout the specification and claims the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “an enzyme” includes mixtures of two or more such enzymes, reference to “the probiotic” includes mixtures of two or more such probiotics, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. “About” can mean within 5% of the stated value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “5” is disclosed, then “about 5” is also disclosed.

Disclosed herein are fragments or variants of polypeptides, and any combination thereof. A protein is a polypeptide. “Fragments” of polypeptides include proteolytic fragments, as well as deletion fragments, in addition to specific antibody fragments discussed elsewhere herein, but do not include the naturally occurring full-length polypeptide (or mature polypeptide). “Variants” of polypeptide binding domains or binding molecules of the present invention include polypeptides comprising one or more amino acid substitutions, insertions, and/or deletions compared to a reference or naturally occurring sequence. Non-naturally occurring variants can be produced using art-known mutagenesis techniques. Variant polypeptides can comprise conservative or non-conservative amino acid substitutions, deletions or additions. It is understood and herein contemplated that any “fragment” or “variant” when referring to polypeptide binding domains or binding molecules of the present invention include any polypeptides which retain at least some of the properties (e.g., coagulation activity for an FVIII variant or fragment, or FVIII binding activity for the vWF fragment, or recycling activity by an albumin fragment) of the reference polypeptide such that a prolonged half-life of FVIII in the coagulation factor complex is obtained.

“Factor VIII”, also referred to herein as “FVIII” is a blood glycoprotein involved in hemostasis. As such, Factor VIII is a coagulation factor. The naturally occurring human FVIII comprises 2,351 amino acids that are processed into multiple forms ranging from 170,000 to 280,000 daltons in molecular weight. There are over 2,000 known mutations. Such mutations provide examples of variants of FVIII. These may be purified from plasma or produced by recombinant DNA techniques. FVIII may be full length, B-region deleted, single chain or other variations as shown herein or known in the art (Lieuw, J. Blood Medicine, 2017:8, 67-73 (incorporated by reference in its entirety for its teachings concerning FVIIIs).

TABLE 1
Available FVIII products

| | | | | | Date of US FDA |
|---|---|---|---|---|---|
| Generation | Products | FVIII | Technology | Half-life* | approval |
| Plasma derived | Antihemophilic factor | Full length | Pooled human plasma | 14.8-17.5 hours | 1966 (Hemofil M), |
| | (Hemofil M ®, Koate- | | | | 1974 (Koate-DVI) |
| | DVI, Monarc-M ®, | | | | |
| | Monoclate-P ®) | | | | |
| Plasma derived/VWF | Antihemophilic | Full length with VWF | Pooled human plasma | 12.2-17.9 hours | 1978 (Alphanate), |
| complex | factor/VWF complex | | | | 1986 (Humate-P), |
| | (Alphanate ®, Humate-P ®, | | | | August 2009 (Wilate) |
| | Wilate ®) | | | | |
| Recombinant: first | Antihemophilic | Full length | BSA in culture and | 14.6 ± 4.9 hours | December 1992 |
| generation | factor recombinant | | human albumin as | | |
| | (Recombinate ®) | | stabilizer | | |
| Recombinant: second | rFVIII-FS (Helixate ®, | Full length | Human plasma protein | 13.74 hours | June 2000 |
| generation | Kogenate) | | solution in culture | | |
| Recombinant: third | Antihemophilic factor | Full length | No human or animal | 12-14.2 hours | July 2003 (Advate), |
| generation | recombinant (Advate ®, | | protein added | | March 2016 (Kovaltry) |
| | Kovaltry ®) | | | | |
| Recombinant: second | Moroctocog alfa | BDD | Human plasma protein | 14.5 ± 5.3 hours | March 2000 |
| generation | (ReFacto ®) | | solution in culture | | |
| Recombinant: third | Moroctocog alfa | BDD | No human or animal | 10.8-12 hours | February 2008 |
| generation | (Xyntha ®), Turoctocog | | protein added | | (Xyntha), October |
| | alfa (Novoeight ®) | | | | 2013 (Novoeight) |
| Recombinant: fourth | Simoctocog alfa | BDD | HEK cells to allow | 17.1 ± 11.2 hours | September 2015 |
| generation | (Nuwiq ®) | | human glycosylation | | |
| Recombinant: third- | Octocog alfa pegol | BDD-PEGylated | PEGylation to parent | 14.69 ± 3.79 hours | December 2016 |
| generation EHL | (Adynovate ®) | | drug Advate | | |
| Recombinant: fourth- | rFVIII-Fc (Eloctate ®) | BDD-rFVIII-FC | HEK cells to allow | 19.7 ± 2.3 hours | June 2014 |
| generation EHL | | | human glycosylation | | |
| Recombinant: third- | rFVIII-SC (Afstyla ®) | EHL single chain | No human or animal | 14.2 hours | May 2016 |
| generation EHL | | | protein added | | |

Note:
*The half-life of the different factors was taken from the product brochures from the manufacturers and differs in how it was determined.
Abbreviations:
FVIII, factor VIII;
US FDA, US Food and Drug Administration;
VWF, Von Willebrand factor;
BSA, bovine serum albumin;
rFVIII, recombinant factor VIII;
BDD, B domain deleted;
HEK, human embryonic kidney;
EHL, extended half-life;
PEG, polyethylene glycol;
SC, single chain;
rFVIII-FS, recombinant FVIII formulated with sucrose;
rFVIII-Fc, antihemophilic factor (recombinant), Fc fusion protein;
rFVIII-SC, antihemophilic factor (recombinant), single chain.

Thus, in one aspect, disclosed herein are “factor VIII coagulation factor fragments” and “factor VIII coagulation factor variants” that retain the coagulation activity of FVIII. As used herein, a “FVIII coagulation factor fragment” refers to FVIII comprising an amino acid sequence that is truncated relative to a full-length FVIII sequences. FVIII coagulation factor fragments include proteolytic fragments, as well as deletion fragments, in addition to specific antibody fragments discussed elsewhere herein, but do not include the naturally occurring full-length polypeptide (or mature polypeptide). “FVIII coagulation factor variants” refers to FVIII polypeptides, including the naturally occurring full-length polypeptide (or mature polypeptide), or fragments thereof, comprising one or more amino acid substitutions, insertions, and/or deletions compared to a reference sequence.

The term “albumin fragment” as used herein means any albumin polypeptide comprising less than full-length albumin while retaining the ability to prolong the half-life of the fusion protein and coagulation factor complex, as described herein (for example a fragment of SEQ ID NO: 8 which comprises amino acid residues 25-609 of albumin of the translated protein, as well as amino acids 1-24 which constitute the signal sequence are cleaved during translation). The disclosed albumin and albumin fragments can comprise cysteine 34 which is a free sulfhydryl in albumin. The term “albumin variant” refers to any albumin polypeptide, including full-length albumin and fragments thereof, which comprises one or more insertions, substitutions, or deletions of relative to SEQ ID NO: 8 that retain the ability to prolong the half-life of the fusion protein.

When paired with a D′D3 or D′D3short either lacking any free cysteines due to substitution or free cysteines not being present, cysteine 34 of albumin can be the only free sulfhydryl. Albumin fragments and variants are known to those of skill in the art. For example, Otagiri et al (2009), Biol. Pharm, Bull. 32(4), 527-534, discloses that 77 albumin variants are known, of these 25 have mutations in domain III. A natural fragment lacking the C-terminal 175 amino acids at the carboxy terminus has been shown to have a reduced half-life (Andersen et al (2010), Clinical Biochemistry 43, 367-372). Iwao et al (2007) studied the half-life of naturally occurring human albumin fragments and variants using a mouse model, and found that K541E and K560E had reduced half-life, E501K and E570K had increased half-life and K573E had almost no effect on half-life (Iwao, et al (2007) B.B.A. Proteins and Proteomics 1774, 1582-1590). Galliano et al (1993) Biochim. Biophys. Acta 1225, 27-32 discloses a natural variant E505K. Minchiotti et al (1990) discloses a natural variant K536E. Minchiotti et al (1987) Biochim Biophys. Acta 916, 41 1-418 discloses a natural variant K574N. Takahashi et al (1987) Proc. Natl. Acad. Sci. USA 84, 4413-4417, discloses a natural variant D550G. Carlson et al. (1992). Proc. Nat. Acad. Sci. USA 89, 8225-8229, discloses a natural variant D550A. These are all incorporated by reference in their entirety for their teachings concerning albumin fragments and variants.

“Von Willebrand Factor,” also referred to herein as “vWF,” is a blood glycoprotein involved in hemostasis. The basic full length vWF monomer is a 2050-amino acid protein. Every monomer contains a number of specific domains with a specific function, including the D′D3 domain which binds to factor VIII (Von Willebrand factor type D domain) and comprises residues 764-1270 (as set forth in SEQ ID NO: 3), the A1 domain responsible for binding platelets and heparin, the A2 domain, the A3 domain that binds collagen, the D4 domain, the B1 domain, the B2 domain, the B3 domain, the C1 domain, the C2 domain, and the cysteine knot domain.

The term “endogenous vWF” as used herein indicates vWF molecules naturally present in plasma. The endogenous vWF molecule can be multimer, but can also be a monomer or a dimer. Endogenous vWF in plasma binds to FVIII and forms a non-covalent complex with FVIII.

The term “vWF fragment” or “vWF fragments” or “vWF variant or VWF variants” used herein means any vWF fragments or variants that interact with FVIII and retain the ability to prolong the half-life of FVIII. The vWF fragment or variant can retain at least one or more properties that are normally provided to FVIII by full-length vWF, e.g., preventing, inhibiting, and/or reducing premature activation to FVIIIa, preventing, inhibiting, and/or reducing premature proteolysis, preventing, inhibiting, and/or reducing association with phospholipid membranes that could lead to premature clearance, preventing, inhibiting, and/or reducing binding to FVIII clearance receptors that can bind naked FVIII but not vWF-bound FVIII, and/or stabilizing the FVIII heavy chain and light chain interactions. The term “vWF fragment” as used herein does not include full length—or mature vWF protein. In a particular embodiment, the “vWF fragment” as used herein comprises a D′ domain and a D3 domain of the VWF protein, but does not include the A1 domain, the A2 domain, the A3 domain, the D4 domain, the B1 domain, the B2 domain, the B3 domain, the CI domain, the C2 domain, and the CK domain of the vWF protein. That is, the vWF fragment can comprise any of the residues from serine 764 through phenylalanine 1270 of vWF including, but not limited to all the residues between serine 764 through phenylalanine 1270. It is understood and herein contemplated that there are embodiments where vWF fragment comprises less than the full D′D3 domain (i.e., less than serine 764 through phenylalanine 1270) referred to herein as “D′D3short.” In one particular embodiment, the “vWF fragment” as used herein comprises a D′D3short. It is contemplated herein that the disclosed D′D3, D′ or D′D3short fragments disclosed herein can further comprise variants (i.e, an amino acid substitution, deletion, or insertion) of the D′D3, D′, or D′D3short amino acid sequence that retain the ability to bind FVIII and prolong the FVIII half-life.

As stated above, D′D3short can comprise or consist of any portion of the D′D3 sequence less than the entire D′D3 sequence from serine 764 through phenylalanine 1270 of vWF (as set forth in SEQ ID NO: 3). Thus, in one particular embodiment The D′D3short does not include a complete D3 domain (SEQ ID NO: 5). Alternatively D′D3short does not contain any portion of the D3 domain, (e.g. D′ only as set forth in SEQ ID NO: 4)), and also does not contain the A1 domain, the A2 domain, the A3 domain, the D4 domain, the B1 domain, the B2 domain, the B3 domain, the CI domain, the C2 domain, and the CK domain of the vWF protein. That is, D′D3short can comprise at least 97 contiguous amino acids of the D′ domain including cysteine 863, and variants thereof, so long is it retains the ability to bind FVIII and prolong the FVIII half-life, especially in vivo, but less than the complete D′D3. Examples of D′D3short domain can include, but are not limited to serine764 through cysteine 1031, serine 764 through asparagine 864 (the full D′ domain as set forth in SEQ ID NO: 4), serine 764 through cysteine 863, leucine 765 through cysteine 863, leucine 765 through asparagine 864, serine 766 through cysteine 863, serine 766 through asparagine 864, serine 764 though arginine 1035, serine 764 though lysine 1036, serine 764 through serine 900, serine 764 through cysteine 1099, serine 764 through cysteine 1142, serine 764 through proline 1197 and serine 764 through proline 1240. Other vWF fragments and variants are known to those of skill in the art and are disclosed herein.

It is understood and contemplated that D′D3 or D′D3short typically binds FVIII via non-covalent bonds, but could also be directly bound to FVIII by a cleavable covalent bond via linkers disclosed herein.

It is understood and herein contemplated that the D′D3 region of vWF has a large number of disulfide bridges and seven unpaired cysteines which reside in the D3 domain and are not present in the D′ domain (serine 764 through asparagine 864). Cysteines at residues 889 and 898 are understood in the art to be important for synthesis and secretion. Cysteines at residues 1099 and 1142 are important for dimerization. The remaining three unpaired cysteines at residues 1222, 1225, and 1227 are variably paired with other cysteines in the chain. The number of unpaired cysteines means there are multiple free sulfhydryls. Replacement of one or more of the D′D3 cysteines with alanines or D′D3short constructs without these cysteines can improve or eliminate this problem and leave the cysteine corresponding to cys34 of albumin as the only free sulfhydryl. Accordingly, disclosed herein are D′D3 or D′D3short fragments wherein the unpaired cysteines (i.e., cysteines at residues 889, 898, 1099, 1142, 1222, 1225, and/or 1227) has been eliminated or substituted for alanine as appropriate for the D′D3 or D′D3short fragment. In some aspects the D′D3short fragment does not comprise any of residues 889, 898, 1099, 1142, 1222, 1225, or 1227 and thus has no available free sulfhydryls. This means that when paired with an albumin or albumin fragment, the only available sulfhydryl is in the albumin fragment corresponding to cys34. Such a construct has the advantage of a highly homogeneous product and providing a single site for adding a linker, label or tag.

A “fusion” or “chimeric” protein can comprise a first amino acid sequence linked to a second amino acid sequence with which it is not naturally linked in nature. In one embodiment, the term “fusion protein,” as used herein, in one example refers to the fusion of the von Willebrand's factor fragment, e.g. D′D3 or D′D3short to albumin, an albumin fragment, an Fc domain, or an Fc fragment. This fusion could for example be accomplished via a genetic construct producing a peptide bond or another type of covalent linkage such as a disulfide bond.

Disclosed herein is a “modifying molecule.” A modifying molecule is any molecule capable of modifying a coagulation factor so that it may interact with a fusion protein while retaining the coagulation enhancing activity of the coagulation factor, e.g. factor VIII (FVIII). For example, a FVIII can be modified with a polyethylene glycol chain and capped by a fatty acid. Various examples of modifying molecules are discussed herein.

A “modified coagulation factor” refers to a coagulation factor which has been modified by a modifying molecule so that it is capable of interacting with a fusion protein while retaining sufficient coagulation activity. The modified coagulation factor can also be referred to as a derivatized FVIII herein. The modified coagulation factor can, for example, be bound to a fusion protein of D′D3 or D′D3short attached by an appropriately sized linker to human albumin in such a way that albumin can bind the fatty acid attached to the modified FVIII or be linked covalently using crosslinking agents to form the modified coagulation factor complex, for example the Factor VIII complex of the subject invention. A modified coagulation factor can also include a genetically modified factor such as by adding a new, additional coding sequence to the FVIII coding sequence, such as an Fc sequence such that it can bind D′D3short containing fusion protein.

As used herein, the term “half-life” refers to a biological half-life of a particular polypeptide in vivo. Half-life may be represented by the time required for half the quantity administered to a subject to be cleared from the circulation and/or other tissues in the animal.

The term “half-life limiting factor” or “FVIII half-life limiting factor” as used herein indicates a factor that prevents the half-life of a FVIII protein from being longer than 1.5 fold or 2 fold compared to wild-type FVIII. For example, full length or mature vWF can act as a FVIII half-life limiting factor by inducing the FVIII and vWF complex to be cleared from the system by one or more vWF clearance pathways. In one example, endogenous vWF is a FVIII half-life limiting factor. In another example, a full-length recombinant vWF molecule non-covalently bound to a FVIII protein can be a FVIII-half-life limiting factor.

The terms “interacts with” or “linked to” as used herein refers in one embodiment to a covalent or non-covalent linkage. The term “covalently linked” or “covalent linkage” refers, for example, to a covalent bond, e.g., a disulfide bond, a peptide bond, or one or more amino acids. In another embodiment “interacts with” or “linked to” means the proteins or protein fragments disclosed herein are connected by a linker between the two proteins or protein fragments that are linked together, for example, between the FVIII and the albumin, and/or between the D′D3 or a D′D3short and albumin, typically via covalent bonds. The first amino acid can be directly joined or juxtaposed to the second amino acid or alternatively an intervening sequence can covalently join the first sequence to the second sequence. The term “linked” can mean not only a fusion of a first amino acid sequence to a second amino acid sequence at the C-terminus or the N-terminus, but also includes insertion of the whole first amino acid sequence (or the second amino acid sequence) into any two amino acids in the second amino acid sequence (or the first amino acid sequence, respectively). In one embodiment, the first amino acid sequence can be joined to a second amino acid sequence by a peptide bond or a linker. As used herein, the linker can be a peptide or a polypeptide or any chemical moiety, for example a disulfide bond or click chemistry bond. When the linker is a peptide or polypeptide, the linker can be any suitable peptide or polypeptide linker known in the art including, but not limited to, a (Gly₄Ser)ₙ linker, a (Gly₃Ser)ₙ linker, a (Gly₂Ser₄)ₙ linker, a (Gly₄Ser₂)ₙ linker, a (GlySer₅)ₙ linker, a (Gly)₆ linker, a (Gly)₈ linker, a GSAGSAAGSGEF linker, and a GGLTPRGVRLGGGSGGGSGGGSEGGGSEGGGSEGGGSEGGGSEGGGSEGGGSGGG SGSGGLTPRGVRL linker (SEQ ID NO: 7), a KLTPRGVRLC linker, or a GGSGGSLTPRGVLGGSWGGSC linker, where n represents 1, 2, 3, 4, 5, 6, 7, 8, 9, 10, 11, 12, 13, 14, 15, 16, 17, 18, 19, 20, 25, or any number of repeats. Additionally, it is contemplated herein that the linker can be cleavable by thrombin, for example, the linker can comprise a thrombin cleavage sequence such as LTPRGVRL (SEQ ID NO: 9). One or both of these thrombin sites could also be substituted with factor Xa cleavage sites, substituting the sequence IDGR or IEGR (SEQ ID NO: 10) for the LTPRGVRL.

The coagulation factor complexes disclosed herein can be used prophylactically. As used herein the term “prophylactic treatment” refers to the administration of a molecule prior to a bleeding episode or consistently during normal activity to prevent, inhibit, or reduce a bleeding episode. In one embodiment, the subject in need of a general hemostatic agent is undergoing, or is about to undergo, surgery. The coagulation factor complex can be administered prior to or after surgery as a prophylactic. The coagulation factor complex can be administered during or after surgery to control an acute bleeding episode. The surgery can include, but is not limited to, liver transplantation, liver resection, dental procedures, or stem cell transplantation.

The coagulation factor complexes of the invention can also be used for on-demand (also referred to as “episodic”) treatment. The term “on-demand treatment” or “episodic treatment” refers to the administration of a chimeric molecule in response to symptoms of a bleeding episode or before an activity that may cause bleeding. In one aspect, the on-demand (episodic) treatment can be given to a subject when bleeding starts, such as after an injury, or when bleeding is expected, such as before surgery. In another aspect, the on-demand treatment can be given prior to activities that increase the risk of bleeding, such as contact sports.

As used herein the term “acute bleeding” refers to a bleeding episode regardless of the underlying cause. For example, a subject may have trauma, uremia, a hereditary bleeding disorder (e.g., factor VII deficiency) a platelet disorder, or resistance owing to the development of antibodies to clotting factors.

Treat, treatment, treating, as used herein refers to, e.g., the reduction in severity of a disease or condition; the reduction in the duration of a disease course; the amelioration of one or more symptoms associated with a disease or condition; the provision of beneficial effects to a subject with a disease or condition, without necessarily curing the disease or condition, or the prophylaxis of one or more symptoms associated with a disease or condition.

In one embodiment, the term “treating” or “treatment” means maintaining a FVIII trough level at least about 1 IU/dL, 2 IU/dL, 3 IU/dL, 4 IU/dL, 5 IU/dL, 6 IU/dL, 7 IU/dL, 8 IU/dL, 9 IU/dL, 10 IU/dL, 11 IU/dL, 12 IU/dL, 13 IU/dL, 14 IU/dL, 15 IU/dL, 16 IU/dL, 17 IU/dL, 18 IU/dL, 19 IU/dL, or 20 IU/dL in a subject by administering a coagulation factor complex of the invention. In another embodiment, treating or treatment means maintaining a FVIII trough level between about 1 and about 20 IU/dL, about 2 and about 20 IU/dL, about 3 and about 20 IU/dL, about 4 and about 20 IU/dL, about 5 and about 20 IU/dL, about 6 and about 20 IU/dL, about 7 and about 20 IU/dL, about 8 and about 20 IU/dL, about 9 and about 20 IU/dL, or about 10 and about 20 IU/dL. Treatment or treating of a disease or condition can also include maintaining FVIII activity in a subject at a level comparable to at least about 1%, 2%, 3%, 4%, 5%, 6%, 7%, 8%, 9%, 10%, 11%, 12%, 13%, 14%, 15%, 16%, 17%, 18%, 19%, or 20% of the FVIII activity in a non-hemophiliac subject. The minimum trough level required for treatment can be measured by one or more known methods and can be adjusted (increased or decreased) for each person.

Coagulation Factor Complexes

Human albumin has a series of properties that are useful in the construction of fusion proteins described herein. It has the ability to prolong the half life of fusion proteins by binding to the neonatal Fc receptor and it binds a number of small molecules including fatty acids and bilirubin. In addition, it has a single exposed sulfhydryl group that can be utilized to attach various ligands. As utilized herein these properties allow the construction of an extended half life, subcutaneously available FVIII. The coagulation factor complexes disclosed herein comprise: a coagulation factor or modified coagulation factor; a fusion protein comprising a first protein fused to albumin, an albumin fragment, an immunoglobulin Fc region, or Fc fragment; and a modifying molecule, wherein the modifying molecule is coupled to the coagulation factor in such a way as to allow binding by the fusion protein, thereby creating a modified coagulation factor; wherein the modified coagulation factor and the fusion protein interact in at least two independent sites, for example a covalent click chemistry linkage site between the modified coagulation factor, e.g. FVIII and an albumin of the fusion protein, and a second non-covalent interaction site between the D′D3short of the fusion protein and the FVIII. The modifying molecule can also be coupled to the fusion protein in such a way as to allow binding of the fusion protein to the coagulation factor or to cause a chemical reaction between the coagulation factor and the fusion protein. The combination of the coagulation factor with the modifying molecule can be referred to as a modified coagulation factor, e.g., modified Factor VIII, herein.

In particular embodiments, disclosed herein is a fusion protein comprising a D′D3short fragment of von Willebrand's factor, fused to full length albumin, or an albumin fragment or a variant thereof, and/or an immunoglobulin Fc domain, or an Fc fragment or variant. The fusion protein can comprise a D′D3short fragment of von Willebrand's factor fused to full length albumin or an albumin fragment or variant. The fusion can be a covalent bond, e.g. a peptide bond. The fusion protein can be covalently bound to a FVIII coagulation factor via a linker comprising click chemistry moieties. The fusion proteins above can include in a pharmaceutical carrier. The fusion proteins can be used to treat hemophilia A. The fusion protein can also be covalently bound to a FVIII coagulation factor via a linker further comprising a cleavable amino acid. The covalent bond between FVIII and the fusion protein can occur via cys34 of albumin.

Disclosed herein is a specific fusion protein referred to herein as “CM110short or CM110s.” CM110 comprises the D′D3 domain or any of the D′D3short fragments of human von Willebrand Factor as disclosed herein. CM110 further comprises a linker (such as, for example, a 56 amino acid glycine serine rich linker as set forth in SEQ ID NO: 7) and a full length human albumin, a albumin variant or fragment thereof, and/or an immunoglobulin Fc domain, or an Fc variant or fragment thereof.

The linker length can, for example, range from 10 amino acids to 100 amino acids, and so, for example, can be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58. 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids in length. Examples of linkers can include, for example, a (Gly₄Ser)ₙ linker, a (Gly₃Ser)ₙ linker, a (Gly₂Ser₄)ₙ linker, a (Gly₄Ser₂)ₙ linker, a (GlySer₅)ₙ linker, a (Gly)₆ linker, a (Gly)₈ linker, a GSAGSAAGSGEF linker, and a GGLTPRGVRLGGGSGGGSGGGSEGGGSEGGGSEGGGSEGGGSEGGGSEGGGSGGG SGSGGLTPRGVRL linker, a KLTPRGVRLC linker, a GGSGGSLTPRGVLGGSWGGSC linker and wherein n represents 1, 2, 3, 4, 5, 6, 7, 8, 9, 10, 11, 12, 13, 14, 15, 16, 17, 18, 19, 20, 25 repeats.

To distinguish a CM110 comprising D′D3 and a D′D3short fragment, CM110short is used to refer to CM110s comprising a D′D3short fragment. One particular example of a CM110short (CM110short764-863LWA) is set forth in SEQ ID NO: 6 which comprises amino acids 1-22 of the human von Willebrand factor, a D′D3short comprising and serine764 through cystein863, a 56 amino acid linker (SEQ ID NO: 7), amino acids 25-609 of the human albumin (as set forth in SEQ ID NO: 8). As shown in the examples, CM110 or CM110s can be formed from plasmid pCM110 or pCM110s containing amino acids 1-22 and 764-1247, or a pCM110short containing amino acids 1-22 and 764-863, of the human von Willebrand factor, a 56 amino acid linker, amino acids 25-609 of the human albumin, with or without a 6×his tag. Also disclosed herein is the coagulation factor complex CM211. CM211 comprises a CM110, a CM110s (with a D′D3short), such as serine764 through cysteine 1031, serine 764 through asparagine 864 (the full D′ domain as set forth in SEQ ID NO: 4), serine 764 through cysteine 863, leucine 765 through cysteine 863, leucine 765 through asparagine 864, serine 766 through cysteine 863, serine 766 through asparagine 864, serine 764 though arginine 1035, serine 764 though lysine 1036, serine 764 through serine 900, serine 764 through cysteine 1099, serine 764 through cysteine 1142, and serine through proline 1240, and FVIII. Where CM211 is comprised of a CM110s, the CM211 can be referred to as a CM211short or CM211s. The CM211s of this invention can be formed via click chemistry as shown herein. As set forth in the examples, a high protein concentration can aid with efficient ligation of the molecules.

Coagulation Factor

vWF is a very large molecule and circulates as a large multimeric complex of these large molecules (Lenting, Christophe, & Denis, 2015). It is so large that it is ingested into macrophages and digested as a particle. Attempts to engineer a smaller fragment of vWF, called D′D3, that protects FVIII have been successful and fusing this fragment to the Fc region increases its half life dramatically (Yee et al., 2014).

The binding constant for vWF and FVIII is about 0.3 nM (Orlova, Kovnir, Vorobiev, Gabibov, & Vorobiev, 2013). The measured binding constant of the D′D3-Fc fusion created by Yee, et al. (Yee et al., 2014) is 1.5 nM. Endogenous FVIII binds tightly but reversibly to vWF such that there is always about 1 to 2 percent of the FVIII free in solution. In both mice and humans, vWF exists at about a fifty-fold higher concentration than FVIII. Between the lower binding constant and the much higher concentration of vWF, FVIII can be quickly competed away from the D′D3-Fc fusion shown in Yee, et al. Blood 124, 445-442, (2014).

One solution to these problems is found by fusing D′D3 or D′D3short to albumin, or an immunoglobulin Fc domain (or any half life prolonging fragment or variant of albumin or the Fc domain), thereby creating a “fusion protein,” as it is referred to herein. Albumin is the most abundant protein in the blood (Peters, 1995). Its 19 day half life in the circulation is determined by its ability to bind to the FcRN, as described herein. It serves two major roles: one is to maintain the osmolarity of the blood and the second is to transport hydrophobic molecules. Albumin is the major transporter of fatty acids. A strategy that has been employed successfully to increase the half life of insulin, for example, is to conjugate insulin to myristic acid, a 14 carbon fatty acid. This molecule is called insulin detemir (Philips & Scheen, 2006). When injected, the fatty acid quickly binds to albumin, increasing the half life of the insulin from 4 minutes to 5 hours.

Immunoglobulins can have a half life extension also mediated by binding to FcRN. Albumin and the Fc region bind to separate sites on FcRN.

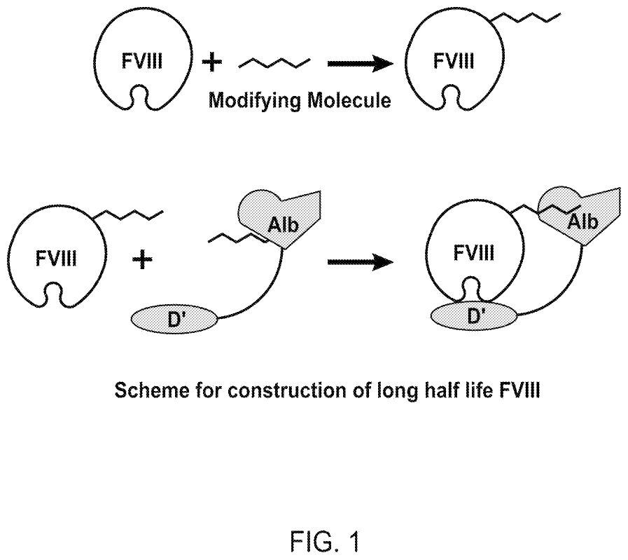

A combination of these two ideas is disclosed herein (FIG. 1) in a novel molecule to solve the long existing half life problem and provide a much needed solution. For example, first, the FVIII can be modified with a polyethylene glycol chain and capped by a fatty acid, for example. This example of a “modifying molecule,” as it is referred to herein, creates an example of a “modified coagulation factor.” The fatty acid thus protrudes from the FVIII molecule in one embodiment to create the modified coagulation factor. The modified coagulation factor can also be referred to as a derivatized FVIII or F8F herein. Next, the modified coagulation factor can be bound to a fusion protein of D′D3 or D′D3short attached by an appropriately sized linker to human albumin, in such a way that albumin can bind the fatty acid attached to the modified FVIII to form the modified coagulation factor complex, for example the Factor VIII or Factor VIIa complex of the subject invention. The D′D3 domain or D′D3short can bind to its cognate site on the modified FVIII and the fatty acid can bind to albumin. The modified coagulation factor can be tethered to the fusion protein at two points, both of which have strong binding constants. In addition, the length of the linker connecting D′D3 or D′D3short to albumin in the fusion protein can be altered, either longer or shorter, such that D′D3 or D′D3short, can be properly or optimally orientated with the Factor VIII binding site for optimal half life extension. In this way, it is much less likely that native vWF will be able to compete effectively for the modified FVIII and attachment to albumin increases the half life substantially.

Another alternative is to genetically modify FVIII by adding an immunoglobulin Fc region to its coding sequence. A second gene coding for the fusion protein containing D′D3short and an Fc region can be transfected into the same cell. The cellular synthetic machinery will then join the modified FVIII and the D′D3short containing fusion protein through sulfhydryl linkage. Such constructs but with D′D3 as a minimum vWF size are disclosed in US Patent Application Publication No. US20150023959 and are incorporated by reference herein. The present invention provided herein, contrary to the teachings in teaches in US20150023959, that the D′D3short, such as D′ without D3, can be substituted for D′D3 and retain effective FVIII binding, e.g non-covalent binding.

The modified coagulation factor and the fusion protein can interact at one, two, three, four, or more sites. In one embodiment, the modified coagulation factor and the fusion protein interact at two independent sites on both molecules. By “independent sites” is meant non-overlapping, or distinct, areas of one, or both, molecules. At least one binding site of the modified coagulation factor can be a natural binding site. In other words, the binding site is naturally occurring on the coagulation factor, and is not part of its modification. The other one or more binding sites on the modified coagulation factor can be modified, such that one or more amino acids in that site is not natural, or native, to the coagulation factor.

The fusion protein can comprise two, three, four, or more proteins fused together. For example, the first fusion protein can comprise a D′D3 domain or D′D3short of von Willebrand's factor. Variants and fragments of vWF are known to those of skill in the art, and are contemplated herein. Examples of such can be found in U.S. Pat. No. 9,125,890, and U.S. Patent Applications 2014/0357564 and 2013/120939. The second protein can comprise albumin, or an immunoglobulin Fc fragment. In one example, the immunoglobulin Fc fragment can comprise a single chain variable region (scFv) specific to the modified coagulation factor. The scFv can be specific to a modified site of the modified coagulation factor.

This dual binding strategy can be accomplished in a number of other ways while still maintaining the required FcRN cycling, as those of skill in the art will appreciate in view of this disclosure. Other ligands can replace the fatty acid in the modifying molecule, since albumin is known to bind a wide variety of ligands, such as bilirubin (Peters, 1995).

Another embodiment is to substitute an antibody/small molecule set for the albumin/fatty acid pair. There are many small molecules that have cognate monoclonal antibodies and these are often used for detection of the small molecule in biological specimens (Bradbury, Sidhu, Dübel, & McCafferty, 2011). A molecule can be constructed that has D′D3short, an amino acid spacer, the Fc region of the immunoglobulins and a single chain variable region, specific for a small molecule, for example, nitrotyrosine. The modifying molecule could then take the form of maleimide-PEG1000-nitrotyrosine. This would have a dual binding effect and FcRN cycling, but using immunoglobulin based recycling.

Another alternative using a similar strategy of modifying FVIII can be used to create a covalent link between the coagulation factor and the fusion protein. Click chemistry or bio-orthogonal chemistry describes molecules that are designed to react only with one another in a complex chemical milieu. A wide variety of these compounds have been developed, some requiring copper catalyst, others operating on strain induced reaction. Unlike using fatty acid binding to establish the second binding site in the complex, these form covalent links that are quite stable in vivo.

The mathematics of intramolecular binding has been described by Kramer and Karpen (Kramer & Karpen, 1998) and in more detail by Zhou (Zhou, 2006). Binding becomes a function of the individual dissociation constants and the effective local concentration. The bond between albumin in the fusion protein and the modified FVIII, for example, tethers the D′D3 or D′D3short fragment to the modified FVIII. The local concentration of the D′D3 or D′D3short fragment then becomes quite high, precluding significant binding of endogenous vWF.

This dual binding strategy overcomes problems encountered by Yee, et al. (Yee et al., 2014). The new molecule created by binding of the FVIII to the fusion protein, referred to herein as the modified coagulation factor complex, has several desirable features not afforded by either FVIII or other long half life FVIII molecules. First, the very tight or covalent binding ensures that there is very little dissociation of the modified FVIII from the fusion protein, preventing loss of the administered FVIII into the large pool of normal vWF. Second, by divorcing the modified coagulation factor complex from the endogenous vWF and using the fusion protein to extend the half life, a much longer half life can be obtained. Third, by administering the modified coagulation complex rather than free FVIII, it can reduce the incidence of inhibitor formation. Fourth, by attaching the albumin to the fusion protein, rather than directly to the FVIII, the FVIII activity is preserved. Direct fusion of albumin to FVIII results in an inactive molecule (Powell, 2014) Positioning albumin away from direct contact with the FVIII should assist efficient recycling by the FcRN. Fifth, the modified FVIII can be administered subcutaneously. Sixth, D′D3short provides advantages in forming coagulation factor complexes. Finally, this can be an entirely human protein produced in a human cell system, which can further reduce the incidence of inhibitor formation.

The half-life of the coagulation factor complex comprising the modified coagulation factor VIII can be at least 10, 20, 30, 40, 50, 60, 70, 80, 90, or 100% or greater compared to a coagulation factor alone. The coagulation factor complex can also have a half-life that is 2, 3, 4, 5, 6, 7, 8, 9, or 10 or more times longer, when compared to an unmodified coagulation factor. More specifically, the half-life of the coagulation factor complex can be at least about 1.5 times, at least about 2 times, at least about 2.5 times, at least about 3 times, at least about 4 times, at least about 5 times, at least about 6 times, at least about 7 times, at least about 8 times, at least about 9 times, at least about 10 times, at least about 11 times, or at least about 12 times longer or more than the half-life of a FVIII protein alone. In one embodiment, the half-life of FVIII is about 1.5-fold to about 20-fold, about 1.5 fold to about 15 fold, or about 1.5 fold to about 10 fold longer than the half-life of wild-type FVIII. In another embodiment, the half-life of the FVIII when in the coagulation factor complex is extended about 2-fold to about 10-fold, about 2-fold to about 9-fold, about 2-fold to about 8-fold, about 2-fold to about 7-fold, about 2-fold to about 6-fold, about 2-fold to about 5-fold, about 2-fold to about 4-fold, about 2-fold to about 3-fold, about 2.5-fold to about 10-fold, about 2.5-fold to about 9-fold, about 2.5-fold to about 8-fold, about 2.5-fold to about 7-fold, about 2.5-fold to about 6-fold, about 2.5-fold to about 5-fold, about 2.5-fold to about 4-fold, about 2.5-fold to about 3-fold, about 3-fold to about 10-fold, about 3-fold to about 9-fold, about 3-fold to about 8-fold, about 3-fold to about 7-fold, about 3-fold to about 6-fold, about 3-fold to about 5-fold, about 3-fold to about 4-fold, about 4-fold to about 6 fold, about 5-fold to about 7-fold, or about 6-fold to about 8 fold as compared to wild-type FVIII or a FVIII protein alone. In other embodiments, the half-life of the coagulation factor complex is at least about 17 hours, at least about 18 hours, at least about 19 hours, at least about 20 hours, at least about 21 hours, at least about 22 hours, at least about 23 hours, at least about 24 hours, at least about 25 hours, at least about 26 hours, at least about 27 hours, at least about 28 hours, at least about 29 hours, at least about 30 hours, at least about 31 hours, at least about 32 hours, at least about 33 hours, at least about 34 hours, at least about 35 hours, at least about 36 hours, at least about 48 hours, at least about 60 hours, at least about 72 hours, at least about 84 hours, at least about 96 hours, or at least about 108 hours. In still other embodiments, the half-life of the coagulation factor complex is about 15 hours to about two weeks, about 16 hours to about one week, about 17 hours to about one week, about 18 hours to about one week, about 19 hours to about one week, about 20 hours to about one week, about 21 hours to about one week, about 22 hours to about one week, about 23 hours to about one week, about 24 hours to about one week, about 36 hours to about one week, about 48 hours to about one week, about 60 hours to about one week, about 24 hours to about six days, about 24 hours to about five days, about 24 hours to about four days, about 24 hours to about three days, or about 24 hours to about two days.

In some embodiments, the average half-life of the coagulation factor complex per subject is about 15 hours, about 16 hours, about 17 hours, about 18 hours, about 19 hours, about 20 hours, about 21 hours, about 22 hours, about 23 hours, about 24 hours (1 day), about 25 hours, about 26 hours, about 27 hours, about 28 hours, about 29 hours, about 30 hours, about 31 hours, about 32 hours, about 33 hours, about 34 hours, about 35 hours, about 36 hours, about 40 hours, about 44 hours, about 48 hours (2 days), about 54 hours, about 60 hours, about 72 hours (3 days), about 84 hours, about 96 hours (4 days), about 108 hours, about 120 hours (5 days), about six days, about seven days (one week), about eight days, about nine days, about 10 days, about 11 days, about 12 days, about 13 days, or about 14 days. In one aspect, the half life of the coagulation factor is at least 60 hours, 72 hours (3 days), 84 hours, 96 hours (4 days), 108 hours, 120 hours (5 days), six days, seven days (one week), eight days, nine days, 10 days, 11 days, 12 days, 13 days, or 14 days

Modifying Molecules

“Modifying molecules,” as disclosed herein, can comprise any molecule which modifies a coagulation factor and renders it capable of interacting with a fusion protein. The modifying molecule can, for example, comprise a fatty acid. The modifying molecule can be attached to the modified coagulation factor through a polyethylene glycol chain, for example. A first and a second protein of the fusion protein can be joined together via a linker, for example. The modified coagulation factor can comprise one or more modified amino acids. Additionally, or alternatively, the fusion protein can comprise modified amino acids. For example, coagulation factor complexes of the invention can, in some embodiments, be composed of amino acids joined to each other by peptide bonds or modified peptide bonds, i.e., peptide isosteres, and may contain amino acids other than the 20 gene-encoded amino acids. The polypeptides may be modified by either natural processes, such as post-translational processing, or by chemical modification techniques which are well known in the art. Such modifications are well described in basic texts and in more detailed monographs, as well as in a voluminous research literature.

Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, PROTEINS—STRUCTURE AND MOLECULAR PROPERTIES, 2nd Ed., T. E. Creighton, W. H. Freeman and Company, New York (1993); POST-TRANSLATIONAL COVALENT MODIFICATION OF PROTEINS, B. C. Johnson, Ed., Academic Press, New York; pgs. 1-12 (1983); Seifter et al., Meth. Enzymol. 182:626-646 (1990); Rattan et al., Ann. N.Y. Acad. Sci. 663:48-62 (1992)).

FVIII can be modified by a number of modifying molecules of the general structure maleimide-PEGn-X, shown in Table 1. A variety of such modifying molecules can be used. For example, various modifications of maleimide-PEG are known to those of skill in the art, such as those presented in U.S. Pat. No. 6,828,401, hereby incorporated by reference in its entirety for its disclosure concerning PEG-maleimide derivatives. In the examples of this invention, this modification did not have any observed effect on the activity of FVIII. Examples of modifying molecules can be found in Table 1.

TABLE 1. Molecules Used to Modify FVIII
Maleimide-PEG5000-biotin
Maleimide-PEG1000-fluorescein isothiocyanate
Maleimide-PEG1000-laurate
Maleimide-PEG1000-myristate
Maleimide-PEG4-6-methyl tetrazine
Maleimide-PEG3-trans cyclooctene
Maleimide-PEG9-transcyclooctene
Maleimide-PEG5000-trans cyclooctene
Maleimide-PEG5000-6-methyl tetrazine
Maleimide-PEG3-azide
Maleimide-PEG1000-azide
Fluoresceinyl maleimide

The chemical moieties for modification of the coagulation factor may be selected from water soluble polymers such as polyethylene glycol, ethylene glycol/propylene glycol copolymers, carboxymethylcellulose, dextran, polyvinyl alcohol and the like. The coagulation factors may be modified at random positions within the molecule, or at predetermined positions within the molecule and may include one, two, three or more attached chemical moieties.

The polymer may be of any molecular weight, and may be branched or unbranched. For polyethylene glycol, the preferred molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog). For example, the polyethylene glycol may have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 25,000, 30,000, 35,000, 40,000, 45,000, 50,000, 55,000, 60,000, 65,000, 70,000, 75,000, 80,000, 85,000, 90,000, 95,000, or 100,000 kDa.

As noted above, the polyethylene glycol may have a branched structure. Branched polyethylene glycols are described, for example, in U.S. Pat. No. 5,643,575; Morpurgo et al., Appl. Biochem. Biotechnol. 56:59-72 (1996); Vorobjev et al., Nucleosides Nucleotides 18:2745-2750 (1999); and Caliceti et al., Bioconjug. Chem. 10:638-646 (1999), the disclosures of each of which are incorporated herein by reference.

The polyethylene glycol molecules (or other chemical moieties) should be attached to the protein with consideration of effects on functional or antigenic domains of the protein. There are a number of attachment methods available to those skilled in the art, such as, for example, the method disclosed in EP 0 401 384 (coupling PEG to G-CSF), herein incorporated by reference; see also Malik et al., Exp. Hematol. 20:1028-1035 (1992), reporting pegylation of GM-CSF using tresyl chloride. For example, polyethylene glycol may be covalently bound through amino acid residues via reactive group, such as a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues glutamic acid residues and the C-terminal amino acid residue. Sulfhydryl groups may also be used as a reactive group for attaching the polyethylene glycol molecules. Preferred for therapeutic purposes is attachment at an amino group, such as attachment at the N-terminus or lysine group.

As suggested above, polyethylene glycol may be attached to proteins via linkage to any of a number of amino acid residues. For example, polyethylene glycol can be linked to proteins via covalent bonds to lysine, histidine, aspartic acid, glutamic acid, or cysteine residues. One or more reaction chemistries may be employed to attach polyethylene glycol to specific amino acid residues (e.g., lysine, histidine, aspartic acid, glutamic acid, or cysteine) of the protein or to more than one type of amino acid residue (e.g., lysine, histidine, aspartic acid, glutamic acid, cysteine and combinations thereof) of the protein.

One may specifically desire proteins chemically modified at the N-terminus. Using polyethylene glycol as an illustration of the present composition, one may select from a variety of polyethylene glycol molecules (by molecular weight, branching, etc.), the proportion of polyethylene glycol molecules to protein (polypeptide) molecules in the reaction mix, the type of pegylation reaction to be performed, and the method of obtaining the selected N-terminally pegylated protein. The method of obtaining the N-terminally pegylated preparation (i.e., separating this moiety from other monopegylated moieties if necessary) may be by purification of the N-terminally pegylated material from a population of pegylated protein molecules. Selective proteins chemically modified at the N-terminus modification may be accomplished by reductive alkylation which exploits differential reactivity of different types of primary amino groups (lysine versus the N-terminal) available for derivatization in a particular protein. Under the appropriate reaction conditions, substantially selective derivatization of the protein at the N-terminus with a carbonyl group containing polymer is achieved.

As indicated above, pegylation of the coagulation factors of the invention may be accomplished by any number of means. For example, polyethylene glycol may be attached to the molecule either directly or by an intervening linker. Linkerless systems for attaching polyethylene glycol to proteins are described in Delgado et al., Crit. Rev. Thera. Drug Carrier Sys. 9:249-304 (1992); Francis et al., Intern. J. of Hematol. 68:1-18 (1998); U.S. Pat. Nos. 4,002,531; 5,349,052; WO 95/06058; and WO 98/32466, the disclosures of each of which are incorporated herein by reference.

One system for attaching polyethylene glycol directly to amino acid residues of proteins without an intervening linker employs tresylated MPEG, which is produced by the modification of monmethoxy polyethylene glycol (MPEG) using tresylchloride. Upon reaction of protein with tresylated MPEG, polyethylene glycol is directly attached to amine groups of the protein. Thus, the invention includes protein-polyethylene glycol conjugates produced by reacting proteins of the invention with a polyethylene glycol molecule having a 2,2,2-trifluoreothane sulphonyl group.

Polyethylene glycol can also be attached to proteins using a number of different intervening linkers. For example, U.S. Pat. No. 5,612,460, the entire disclosure of which is incorporated herein by reference, discloses urethane linkers for connecting polyethylene glycol to proteins. Protein-polyethylene glycol conjugates wherein the polyethylene glycol is attached to the protein by a linker can also be produced by reaction of proteins with compounds such as MPEG-succinimidylsuccinate, MPEG activated with 1,1′-carbonyldiimidazole, MPEG-2,4,5-trichloropenylcarbonate, MPEG-p-nitrophenolcarbonate, and various MPEG-succinate derivatives. A number of additional polyethylene glycol derivatives and reaction chemistries for attaching polyethylene glycol to proteins are described in International Publication No. WO 98/32466, the entire disclosure of which is incorporated herein by reference. Pegylated protein products produced using the reaction chemistries set out herein are included within the scope of the invention.

The number of polyethylene glycol moieties attached to a modified coagulation factor of the invention (i.e., the degree of substitution) may also vary. For example, the pegylated proteins of the invention may be linked, on average, to 1, 2, 3, 4, 5, 6, 7, 8, 9, 10, 12, 15, 17, 20, or more polyethylene glycol molecules. Similarly, the average degree of substitution within ranges such as 1-3,2-4, 3-5,4-6, 5-7,6-8, 7-9,8-10, 9-11, 10-12, 11-13, 12-14, 13-15, 14-16, 15-17, 16-18, 17-19, or 18-20 polyethylene glycol moieties per protein molecule. Methods for determining the degree of substitution are discussed, for example, in Delgado et al., Crit. Rev. Thera. Drug Carrier Sys. 9:249-304 (1992).

The polypeptides of the invention can be recovered and purified from chemical synthesis and recombinant cell cultures by standard methods which include, but are not limited to, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification. Well known techniques for refolding protein may be employed to regenerate active conformation when the polypeptide is denatured during isolation and/or purification.

The presence and quantity of modified coagulation factor complexes of the invention may be determined using ELISA, a well known immunoassay known in the art. In one ELISA protocol that would be useful for detecting/quantifying modified molecules of the invention, comprises the steps of coating an ELISA plate with an anti-human serum albumin antibody, blocking the plate to prevent non-specific binding, washing the ELISA plate, adding a solution containing the molecule of the invention (at one or more different concentrations), adding a secondary anti-therapeutic protein specific antibody coupled to a detectable label (as described herein or otherwise known in the art), and detecting the presence of the secondary antibody. In an alternate version of this protocol, the ELISA plate might be coated with the anti-therapeutic protein specific antibody and the labeled secondary reagent might be the anti-human albumin superfamily specific antibody.

Polypeptide and Polynucleotide Fragments and Variants

The present invention is further directed to fragments of the coagulation factor complexes described herein as well as fragments of individual components of the coagulation factor complexes, such as the modified coagulation factor, the modifying molecule, or the fusion protein. These modifications can include those disclosed herein, which modify the molecules in such a way as to increase activity or half life, or other modifications that enhance the properties of the molecule or make it desirable for other reasons.

Even if a deletion of one or more amino acids results in modifications or loss of one or more functions, sufficient coagulation function of the complex may still be retained. Accordingly, fragments of the molecules disclosed herein, include the full length protein as well as polypeptides having one or more residues deleted from the amino acid sequence of the reference polypeptide, are contemplated herein.

The present application is directed to proteins containing polypeptides at least 50%, 55%, 60%, 65%, 70%, 75%, 80%, 85%, 90%, 95%, 96%, 97%, 98% or 99% identical to a reference polypeptide sequence (e.g., a coagulation factor, a modifying molecule, or a fusion protein) set forth herein, or fragments thereof.

“Variant” refers to a polynucleotide or nucleic acid differing from a reference nucleic acid or polypeptide, but retaining essential properties thereof. Generally, variants are overall closely similar, and, in many regions, identical to the reference nucleic acid or polypeptide.

As used herein, “variant” refers to a protein disclosed herein which differs in sequence from the known sequence of the protein, but retains at least one functional and/or therapeutic property thereof (e.g., a therapeutic activity and/or biological activity, including but not limited to coagulation) as described elsewhere herein or otherwise known in the art. Generally, variants are overall very similar, and, in many regions, identical to the amino acid sequence of the protein of interest or albumin superfamily protein.

The present invention is also directed to proteins which comprise, or alternatively consist of, an amino acid sequence which is at least 80%, 85%, 90%, 95%, 96%, 97%, 98%, 99% or 100%, identical to, for example, the amino acid sequence of the coagulation factor itself, the fusion protein, or the modifying molecule. Fragments of these polypeptides are also provided (e.g., those fragments described herein). Further polypeptides encompassed by the invention are polypeptides encoded by polynucleotides which hybridize to the complement of a nucleic acid molecule encoding an amino acid sequence of the invention under stringent hybridization conditions (e.g., hybridization to filter bound DNA in 6 times sodium chloride/sodium citrate (SSC) at about 45 degrees Celsius, followed by one or more washes in 0.2 times SSC, 0.1% SDS at about 50-65 degrees Celsius), under highly stringent conditions (e.g., hybridization to filter bound DNA in 6 times sodium chloride/sodium citrate (SSC) at about 45 degrees Celsius, followed by one or more washes in 0.1 times SSC, 0.2% SDS at about 68 degrees Celsius), or under other stringent hybridization conditions which are known to those of skill in the art (see, for example, Ausubel, F. M. et al., eds., 1989 Current protocol in Molecular Biology, Green publishing associates, Inc., and John Wiley & Sons Inc., New York, at pages 6.3.1-6.3.6 and 2.10.3). Polynucleotides encoding these polypeptides are also encompassed by the invention.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino- or carboxy-terminal positions of the reference amino acid sequence or anywhere between those terminal positions, interspersed either individually among residues in the reference sequence or in one or more contiguous groups within the reference sequence.

TABLE 2
Amino Acid Substitutions
Original Residue Exemplary Conservative
Substitutions, others are known in the art.

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation. For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.
Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.
It is understood that there are numerous amino acid and peptide analogs which can be incorporated into the disclosed compositions. For example, there are numerous D amino acids or amino acids which have a different functional substituent then L amino acids. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way.
Molecules can be produced that resemble peptides, but which are not connected via a natural peptide linkage. For example, linkages for amino acids or amino acid analogs can include CH₂NH—, —CH₂S—, —CH₂—CH₂—CH═CH— (cis and trans), —COCH₂—CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, n) Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others. D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations.

As a practical matter, whether any particular polypeptide is at least 80%, 85%, 90%, 95%, 96%, 97%, 98% or 99% identical to, for instance, the amino acid sequence of a coagulation factor or a fragment, can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci. 6:237-245 (1990)). In a sequence alignment the query and subject sequences are either both nucleotide sequences or both amino acid sequences. The result of said global sequence alignment is expressed as percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter.

The polynucleotide variants of the invention may contain alterations in the coding regions, non-coding regions, or both. Especially preferred are polynucleotide variants containing alterations which produce silent substitutions, additions, or deletions, but do not alter the desired properties or activities of the encoded polypeptide. Nucleotide variants produced by silent substitutions due to the degeneracy of the genetic code are preferred. Moreover, polypeptide variants in which less than 50, less than 40, less than 30, less than 20, less than 10, or 5-50, 5-25, 5-10, 1-5, or 1-2 amino acids are substituted, deleted, or added in any combination can be utilized. Polynucleotide variants can be produced for a variety of reasons, e.g., to optimize codon expression for a particular host (change codons in the human mRNA to those preferred by a bacterial host, such as, yeast or E. coli).

Naturally occurring variants are called “allelic variants,” and refer to one of several alternate forms of a gene occupying a given locus on a chromosome of an organism. (Genes II, Lewin, B., ed., John Wiley & Sons, New York (1985)). These allelic variants can vary at either the polynucleotide and/or polypeptide level and are included in the present invention. Alternatively, non-naturally occurring variants may be produced by mutagenesis techniques or by direct synthesis.

Using known methods of protein engineering and recombinant DNA technology, variants may be generated to improve or alter the characteristics of the polypeptides of the present invention. For example, one or more amino acids can be deleted from the N-terminus or C-terminus of the polypeptide of the present invention without substantial loss of biological function. As an example, Ron et al. (J. Biol. Chem. 268: 2984-2988 (1993)) reported variant KGF proteins having heparin binding activity even after deleting 3, 8, or 27 amino-terminal amino acid residues. Similarly, Interferon gamma exhibited up to ten times higher activity after deleting 8-10 amino acid residues from the carboxy terminus of this protein. (Dobeli et al., J. Biotechnology 7:199-216 (1988).)

Thus, the invention further includes polypeptide variants which have the desired functional activity (e.g., biological activity and/or therapeutic activity). In highly preferred embodiments the invention provides modifications to coagulation factors, which modifications allow for an increased functional activity, such as a prolonged half-life.

Also disclosed are methods of treating a subject with a disease requiring coagulation factor infusion, the method comprising administering to the subject the coagulation factor complex disclosed herein. The disease can be hemophilia A, for example. The administration of the coagulation factor complex to the subject result can result in a blood level half-life of the coagulation factor complex which is greater than the blood level half-life obtained upon administration of the coagulation factor alone. The coagulation factor complex can be administered to the subject via injection (including subcutaneous injection), inhalation, internasally, or orally.

The modified coagulation factor complexes of the invention or formulations thereof may be administered by any conventional method including parenteral (e.g. subcutaneous or intramuscular) injection or intravenous infusion. The treatment may consist of a single dose or a plurality of doses over a period of time.

The coagulation factor complexes disclosed herein can be present as a pharmaceutical formulation, together with one or more acceptable carriers. The carrier(s) must be “acceptable” in the sense of being compatible with the coagulation factor complex, and not deleterious to the recipients thereof.

The formulations may conveniently be presented in unit dosage form and may be prepared by any of the methods well known in the art of pharmacy. Such methods include the step of bringing into association the coagulation factor complex with the carrier that constitutes one or more accessory ingredients. In general the formulations are prepared by uniformly and intimately bringing into association the active ingredient with liquid carriers or finely divided solid carriers or both, and then, if necessary, shaping the product.

Kits

Also disclosed herein are kits comprising the coagulation factor complexes. Formulations or compositions of the invention may be packaged together with, or included in a kit with, instructions or a package insert referring to the extended shelf-life of the coagulation factor complex. For instance, such instructions or package inserts may address recommended storage conditions, such as time, temperature and light, taking into account the extended or prolonged shelf-life of the coagulation factor complexes of the invention. Such instructions or package inserts may also address the particular advantages of the coagulation factor complexes of the inventions, such as the ease of storage for formulations that may require use in the field, outside of controlled hospital, clinic or office conditions. As described above, formulations of the invention may be in aqueous form and may be stored under less than ideal circumstances without significant loss of therapeutic activity.

Methods of Treating

The coagulation factor complexes and/or polynucleotides of the invention may be administered alone or in combination with other therapeutic agents. They may be administered in combination with other coagulation factor complexes and/or polynucleotides of the invention. Combinations may be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents given first, followed by the second.

In specific aspects, coagulation factor complex used in methods of the present invention can be contained in a formulation containing a buffer, a sugar and/or a sugar alcohol (including without limitation trehalose and mannitol), a stabilizer (such as glycine), and a surfactant (such as Polysorbate 80). In further embodiments, the formulation may further include sodium, histidine, calcium, and glutathione.

In one aspect, the formulations comprising the coagulation factor complex are lyophilized prior to administration. Lyophilization is carried out using techniques common in the art and should be optimized for the composition being developed (Tang et al., Pharm Res. 21: 191-200, (2004) and Chang et al, Pharm Res. 13:243-9 (1996).

Methods of preparing pharmaceutical formulations can include one or more of the following steps: adding a stabilizing agent as described herein to said mixture prior to lyophilizing, adding at least one agent selected from a bulking agent, an osmolality regulating agent, an d a surfactant, each of which as described herein, to said mixture prior to lyophilization. A lyophilized formulation is, in one aspect, at least comprised of one or more of a buffer, a bulking agent, and a stabilizer. In this aspect, the utility of a surfactant is evaluated and selected in cases where aggregation during the lyophilization step or during reconstitution becomes an issue. An appropriate buffering agent is included to maintain the formulation within stable zones of pH during lyophilization.

The standard reconstitution practice for lyophilized material is to add back a volume of pure water or sterile water for injection (WFI) (typically equivalent to the volume removed during lyophilization), although dilute solutions of antibacterial agents are sometimes used in the production of pharmaceuticals for parenteral administration (Chen, Drug Development and Industrial Pharmacy, 18: 131 1-1354 (1992)).

The lyophilized material may be reconstituted as an aqueous solution. A variety of aqueous carriers, e.g., sterile water for injection, water with preservatives for multi dose use, or water with appropriate amounts of surfactants (for example, an aqueous suspension that contains the active compound in admixture with excipients suitable for the manufacture of aqueous suspensions). In various aspects, such excipients are suspending agents, for example and without limitation, sodium carboxymethylcellulose, methylcellulose, hydroxypropylmethylcellulose, sodium alginate, polyvinylpyrrolidone, gum tragacanth and gum acacia: dispersing or wetting agents are a naturally-occurring phosphatide, for example and without limitation, lecithin, or condensation products of an alkylene oxide with fatty acids, for example and without limitation, polyoxyethylene stearate, or condensation products of ethylene oxide with long chain aliphatic alcohols, for example and without limitation, heptadecaethyl-eneoxycetanol, or condensation products of ethylene oxide with partial esters derived from fatty acids and a hexitol such as polyoxyethylene sorbitol monooieate, or condensation products of ethylene oxide with partial esters derived from fatty acids and hexitol anhydrides, for example and without limitation, polyethylene sorbitan monooieate. In various aspects, the aqueous suspensions also contain one or more preservatives, for example and without limitation, ethyl, or n-propyl, p-hydroxybenzoate.

In certain embodiments, compositions of the present invention are liquid formulations for administration with the use of a syringe or other storage vessel. In further embodiments, these liquid formulations are produced from lyophilized material described herein reconstituted as an aqueous solution. In a further aspect, the compositions of the invention further comprise one or more pharmaceutically acceptable carriers. The phrases “pharmaceutically” or “pharmacologically” acceptable refer to molecular entities and compositions that are stable, inhibit protein degradation such as aggregation and cleavage products, and in addition do not produce allergic, or other adverse reactions when administered using routes well-known in the art, as described below. “Pharmaceutically acceptable carriers” include any and all clinically useful solvents, dispersion media, coatings, antibacterial and antifungal agents, isotonic and absorption delaying agents and the like, including those agents disclosed above.

Single or multiple administrations of coagulation factor complexes are carried out with the dose levels and pattern being selected by the treating physician. For the prevention, inhibition, reduction, or treatment of disease, the appropriate dosage depends on the type of disease to be treated, the severity and course of the disease, whether drug is administered for preventive or therapeutic purposes, previous therapy, the patient's clinical history and response to the drug, and the discretion of the attending physician.

In further embodiments and in accordance with any of the above, treatment of coagulation diseases such as Hemophilia A may involve an initial treatment of coagulation factor complex alone or in combination with another agent, followed by one or more repeat doses of coagulation factor complex and/or other agents. The nature of the initial and then the subsequent repeat administrations will depend in part on the disease being treated.

In further aspects, coagulation factor complex can be administered to a subject in doses ranging from 0.5 IU/kg-200 IU kg. In some embodiments, coagulation factor complex is administered in doses ranging from 1-190, 5-180, 10-170, 15-160, 20-450, 25-140, 30-130, 35-120, 40-110, 45-100, 50-90, 55-80, or 60-70 IU/kg. In further embodiments and in accordance with any of the above, coagulation factor complex can be administered to a subject at doses of between about 1 IU/kg to about 150 IU/kg. In still further embodiments, the coagulation factor complex is administered at doses of between 1.5 IU/kg to 150 IU/kg, 2 IU/kg to 50 IU/kg, 5 IU/kg to 40 IU/kg, 10 IU/kg to 20 IU/kg, 10 IU/kg to 100 IU kg, 25 IU/kg to 75 IU/kg, and 40 IU kg to 75 IU/kg. In still further embodiments, coagulation factor complex is administered at 2, 5, 7.5, 10, 15, 20, 25, 30, 35, 40, 45, or 50 IU/kg. As will be appreciated and as is discussed further herein, appropriate dosages of coagulation factor complex may be ascertained through use of established assays for determining blood level dosages in conjunction with appropriate dose-response data.

In certain examples, the complexes of the current invention can be infused or adminstered to the muscle to treat hemophilia A. Compositions of coagulation factor complex can be contained in pharmaceutical formulations, as described herein. Such formulations can be administered orally, topically, transdermally, parenterally, by inhalation spray, vaginally, rectally, or by intracranial injection. The term parenteral as used herein includes subcutaneous injections, intravenous, intramuscular, intracisternal injection, or infusion techniques. Administration by subcutaneous, intravenous, intradermal, intramuscular, intramammary, intraperitoneal, intrathecal, retrobulbar, intrapulmonary injection and or surgical implantation at a particular site is contemplated as well. Generally, compositions are essentially free of pyrogens, as well as other impurities that could be harmful to the recipient.

In one aspect, formulations of the invention are administered by an initial bolus followed by a continuous infusion to maintain therapeutic circulating levels of drug product. As another example, the inventive compound is administered as a one-time dose. Those of ordinary’ skill in the art will readily optimize effective dosages and administration regimens as determined by good medical practice and the clinical condition of the individual patient. The route of administration can be, but is not limited to, by intravenous, intraperitoneal, subcutaneous, or intramuscular administration. The frequency of dosing depends on the pharmacokinetic parameters of the agents and the route of administration. The optimal pharmaceutical formulation is determined by one skilled in the art depending upon the route of administration and desired dosage, See for example, Remington's Pharmaceutical Sciences, 18th Ed., 1990, Mack Publishing Co., Easton, Pa. 18042 pages 1435-1712, the disclosure of which is hereby incorporated by reference in its entirety for ail purposes and in particular for ail teachings related to formulations, routes of administration and dosages for pharmaceutical products. Such formulations influence the physical state, stability, rate of in vivo release, and rate of in vivo clearance of the administered agents. Depending on the route of administration, a suitable dose is calculated according to body weight, body surface area or organ size, Appropriate dosages may be ascertained through use of established assays for determining blood level dosages in conjunction with appropriate dose-response data. The final dosage regimen is determined by the attending physician, considering various factors which modify the action of drugs, e.g. the drug's specific activity, the severity of the damage and the responsiveness of the patient, the age, condition, body weight, sex and diet of the patient, the severity of any infection, time of administration and other clinical factors. By way of example, a typical dose of coagulation factor complex of the present invention is approximately 50 IU/kg, equal to 500 μg/kg. As studies are conducted, further information will emerge regarding the appropriate dosage levels and duration of treatment for various diseases and conditions.

In some embodiments, coagulation factor complex is administered to a subject alone. In some embodiments, coagulation factor complex is administered to a subject in combination with one or more other coagulation factors.

In further embodiments, coagulation factor complex is administered to a subject no more than once daily. In further embodiments, coagulation factor complex is administered to a subject: no more than once every other day, no more than once every third day, no more than once every fourth day, no more than once every fifth day, no more than once a week, no more than once every two weeks, no more than once a month. In still further embodiments, coagulation factor complex is administered to a subject no more than twice a day.

Having generally described the invention, the same will be more readily understood by reference to the following examples, which are provided by way of illustration and are not intended as limiting.

Without further description, it is believed that one of ordinary skill in the art can, using the preceding description and the following illustrative examples, make and utilize the alterations detected in the present invention and practice the claimed methods. The following working examples therefore, specifically point out preferred embodiments of the present invention, and are not to be construed as limiting in any way the remainder of the disclosure.

### EXAMPLES

### Example 1. Synthesis and Production of CM110

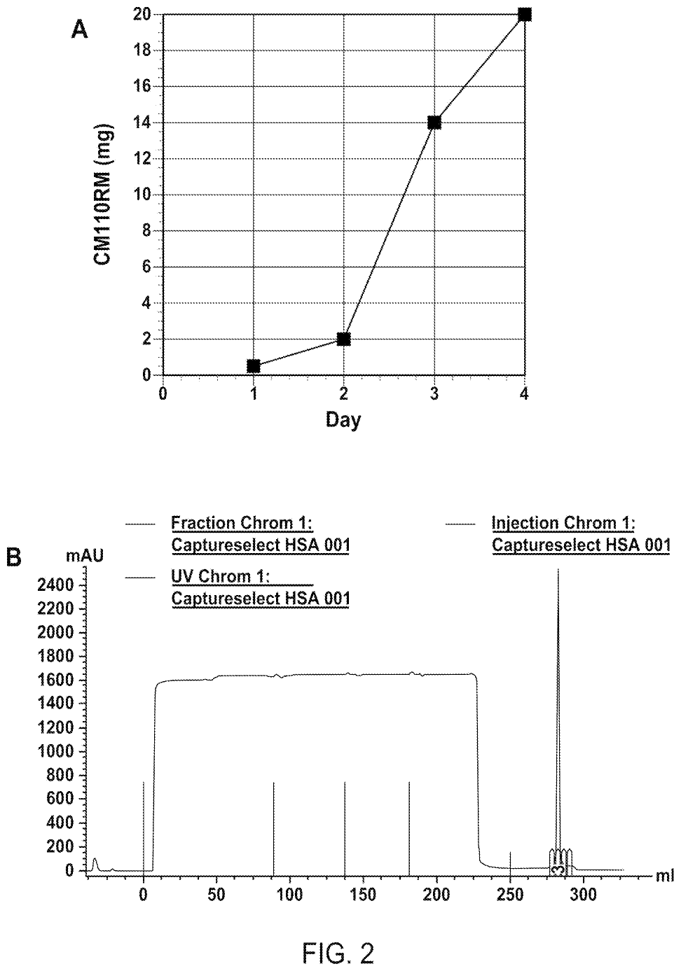

The synthetic protein CM110 is designed to protect FVIII from degradation and nonspecific binding, divorce it from the endogenous vWF and extend its half life in the blood. In a specific embodiment, the protein consists of the D′D3 region of vWF, a 56 amino acid glycine, serine rich linker, and a full length human albumin A codon optimized DNA sequence encoding this protein was synthesized by Gene Art and inserted into the expression plasmid pcDNA3.4TOPO. Transient transfection of this plasmid into the human embryonic kidney cell line Expi293 produced substantial quantities of protein in the cell supernate over four days of culture (FIG. 2A). The protein was purified directly from the supernate using affinity chromatography on an HSA affinity column (FIG. 2B). The protein has a predicted molecular weight of 124,744 and was seen to run at about 125,000 on an SDS polyacrylamide gel (FIG. 2C). When the protein was passed over a calibrated Superdex S-200 gel filtration column the protein exhibited a molecular weight of 250,000, suggesting that in solution, it behaves as a dimer of the 125,000 units (FIG. 2D).

Von Willebrand's factor naturally forms a highly ordered polymer in the Weibel Palade bodies and when secreted travels the blood as a selection of multimers. Two free sulfhydryls in the D′D3 region were utilized in the initial formation of these multimers. These correspond to cysteine 1099 and cysteine 1142 in the native vWF. These cysteines were mutated to alanines in the D′D3 construct to prevent formation of higher polymers.

### Example 2. Labeling of FVIII and CM110 with Click Reagents

Disclosed herein is a method of producing a secondary protein and utilizing the natural ability of FVIII to bind to the D′D3 region of vWF. In this way, the two proteins form the appropriate complex that is then chemically crosslinked. An excess of the D′D3 containing reagent was utilized in order to drive as much of the FVIII into the complex as possible. In such a situation, bifunctional crosslinkers often form polymers, reducing the yield of functional complex. Click chemistry agents are designed to react only with one another, precluding polymer formation. The reaction pair of trans-cyclooctene and methyl tetrazine has the appropriate characteristics of fast, quantitative reactivity.

The D′D3 region of vWF has a large number of disulfide bridges but only a few free sulfhydryls, particularly those corresponding to cys1099 and cys1142. These are important in formation of the ordered polymer that is usually formed by vWF. In the earliest construct, undesirable polymers of CM110 were formed in solution. Replacement of these cysteines with alanines eliminated this problem and left the cysteine corresponding to cys34 of albumin as the only consistently free sulfhydryl. A variety of maleimide based click reagents were tried, but were only able to modify about 50% of the CM110 using this method. Tests of proteins labeled at this cysteine also seemed to ligate very poorly to its labeled counterpart.

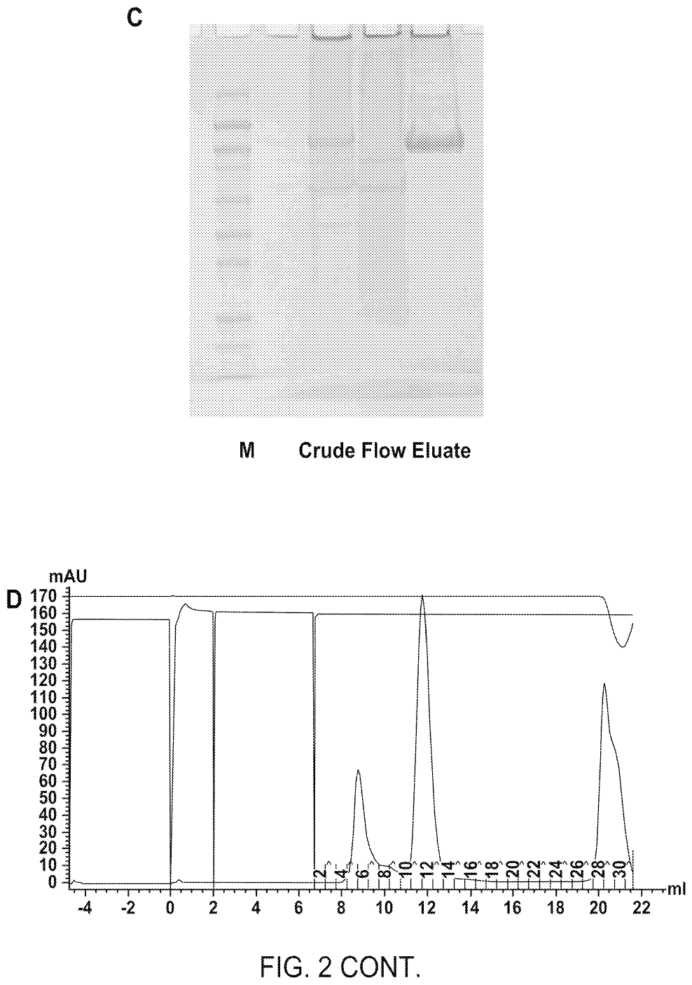

An N-hydroxysuccinimde based reagent, NHS-PEG12-TCO, was used. The length of the PEG polymer can be as small as 3 PEG units or as long as 50 PEG units. The efficiency of ligation was best in this construct around 10 to 15 units. NHS reacts with primary amines and usually modifies several amines in any protein. CM110 was treated with NHS-PEG12-TCO, then treated the labeled protein with BODIPY—tetrazine. FIG. 3A shows that CM110 was efficiently labeled in this way, incorporating about 10 molecules of NHS-PEG12-TCO per 250 kd protein molecule (FIG. 3B).

Treating CM110 with tris-(2 carboxyethyl) phosphine (TCEP) before attempting to label with maleimide based reagents is another route to more efficient ligation. When CM110 was treated for one hour with 100 μM TCEP, then run on a Superdex S-200 Increase column, it emerged at the monomeric molecular weight of 125,000. Labeling with fluoresceinyl maleimide demonstrated that one molecule of fluorescein was incorporated per CM110. Test ligations with maleimide-PEG4-MeTET labeled FVIII and maleimide-PEG3-TCO labeled CM110 suggest substantially better ligation efficiency.

FVIII has three free sulfhydryls, two in the heavy chain and one in the light chain. When FVIII was treated with fluoresceinyl maleimide, it was found that primarily, the heavy chain is labeled (FIG. 3C). Treatment of the labeled FVIII with thrombin demonstrated that both cys310 and cys692 of the heavy chain are labeled.

### Example 3—Formation of CM211

For complex formation, FVIII was labeled with maleimide-PEG4-methyltetrazine (mal-P4-tet). Importantly, labeling had no effect on the activity of FVIII in the Coatest assay. It was found that PEG containing molecules of any size often separated poorly from the labeled protein when run on standard desalting columns so after labeling, FVIII was passed over Superdex S-200 in 20 mM HEPES, pH 7.4, 300 mM NaCl, 4 mM CaCl2.

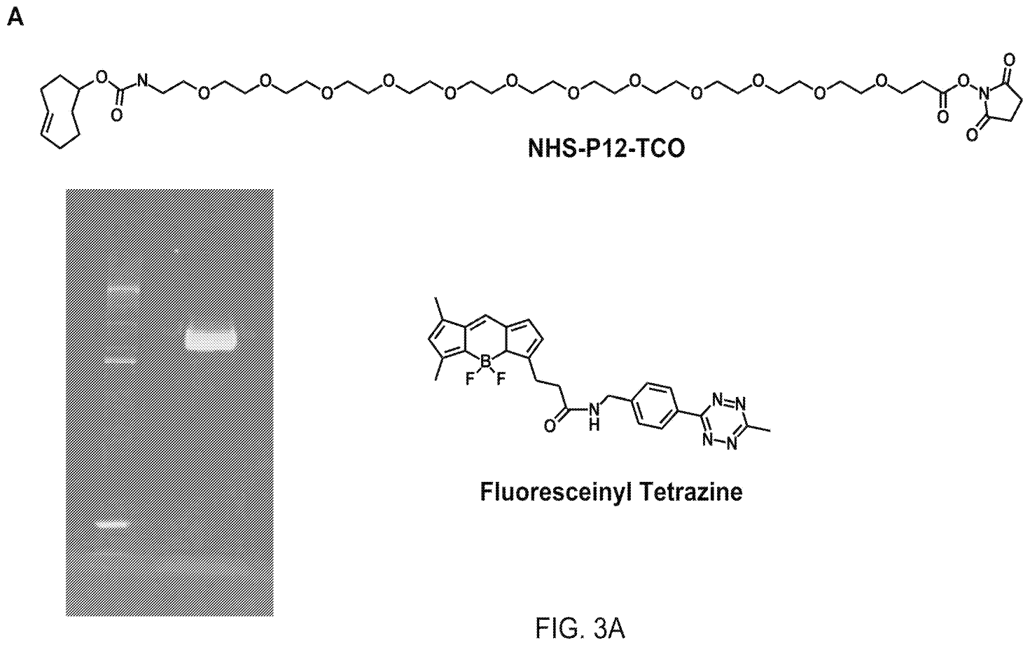

CM110 was labeled with NHS-PEG12-TCO and also filtered on Superdex S-200 in the same buffer. Fractions containing labeled CM110 and labeled FVIII were pooled, diluted 1 to 2 with water to reduce the salt to 150 mM, then concentrated to 200 μl in a spin filter. The spin filter was washed with 100 μl of the same buffer and pooled with the concentrated solution. This produced a solution that was about 5 mg/ml in CM110 and 1 mg/ml in FVIII. A high protein concentration, greater than 1 mg/ml was important for efficient ligation. After incubation at room temperature for 2 hours, the solution was filtered through Superdex S-200 again (FIG. 4A). The complex, now termed CM211, emerged before the free CM110 peak and essentially every unit of FVIII was contained in the complex. From its elution volume on the Superdex S-200 column, CM211 appears to consist of one molecule of FVIII and the CM110 dimer, with a molecular weight of 430,000.

When CM211 is run on an SDS gel, several bands appear (FIG. 4B). The lowest molecular weight bands, around the 80,000 marker, correspond to FVIII. The next highest, is free CM110. The next highest are ligated CM110 and FVIII chains Immunoblotting experiments confirm the presence of both heavy chain and light chain in both the lower and higher molecular weight species, as well as albumin. This showed that CM211 consisted of a mixture of ligated and unligated material, as expected. This can result from overall poor ligation or from an assortment of the various pieces ligated together.

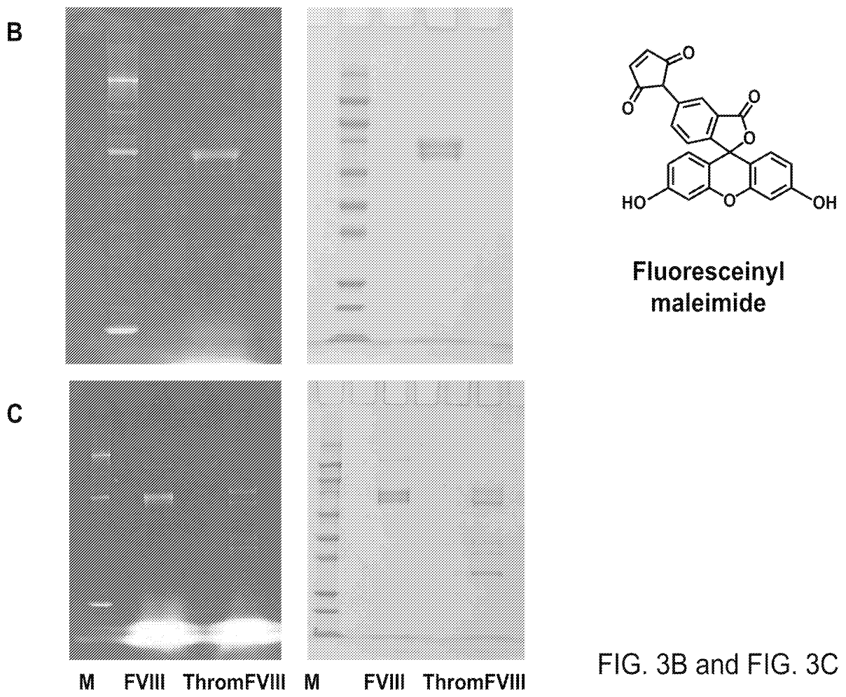

An attempt to resolve this is shown in FIG. 5. In the purification of FVIII from plasma, the FVIII and vWF can be dissociated by the addition of 0.25 M CaCl2. If there is overall poor ligation, treatment of the complex with the CaCl2 should result in the release of free FVIII. When the complex is formed with unlabeled CM110 and unlabeled FVIII, free FVII is released in the presence of the calcium. When CM211 is treated with the high calcium, no free FVIII is released, suggesting that the band heterogeneity is a result of mixed ligation (FIG. 5A). FVIII consists of heavy and light chains but there are multiple bands of each on an SDS gel, presumably due to mixed glycosylation. Likewise, when CM211 is chromatographed in buffer containing 20 mM HEPES, pH 7.4, 0.8 M NaCl, 4 mM CaCl2, no free FVIII is released (FIG. 5B). These are also conditions which disrupt FVIII, vWF binding.

### Example 4—Specific Activity

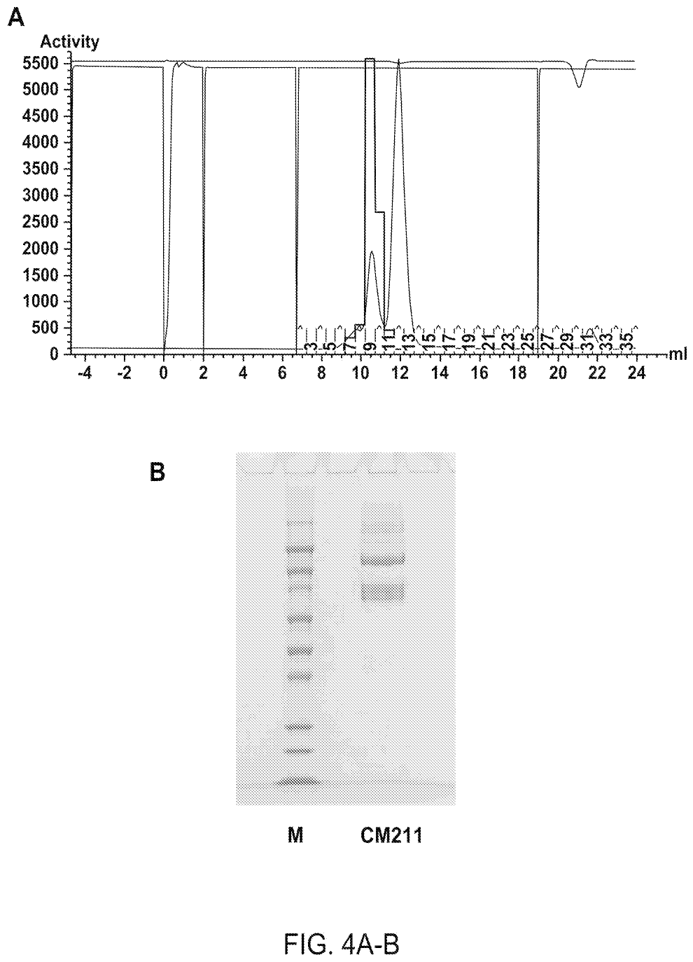

Modification of FVIII in other situations results in a divergence of the specific activity of the molecule in the Coatest assay versus clotting assays, such as the activated partial thromboplastin time (APTT). The Coatest assay is a two stage assay and contains activated factor IX and thrombin, to activate the FVIII. Factor X is then activated proportionally to the amount of activated FVIII by binding of IXa and X. Xa then hydolyzes the chromogenic substrate. The APTT depends on activation and functioning of the entire intrinsic coagulation pathway to form a clot. When measured in the Coatest assay, CM211 has a specific activity of about 8,500 IU/mg, similar to most recombinant FVIII. When measured in the APTT, the specific activity is about 6.5 fold lower, meaning that more Coatest units are required to normalize the APTT in FVIII deficient plasma (FIG. 6A, 6B). The discrepancy between the Coatest units and the APTT units suggest that the D′D3 dissociation from FVIII is slow, as expected in the design of this molecule. The specific activity was therefore defined according to the APTT assay in the thrombin generation assay and for experiments in mice.

### Example 5—Thrombin Generation

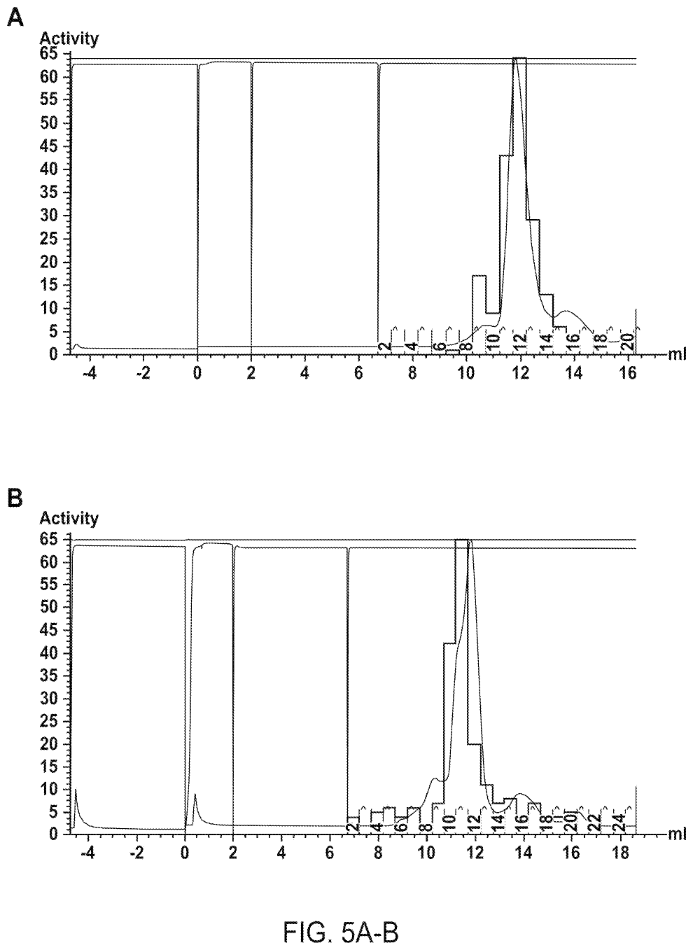

Thrombin generation is the main objective of the coagulation cascade. CM211 was added at various concentrations to FVIII-deficient plasma to measure thrombin generation. Using the specific activity determined in the APTT assay, the appropriate specific activity corrects thrombin generation as measured by both thrombin generation itself and by the area under the curve (FIG. 7A, 7B).

### Example 6—Mouse Studies

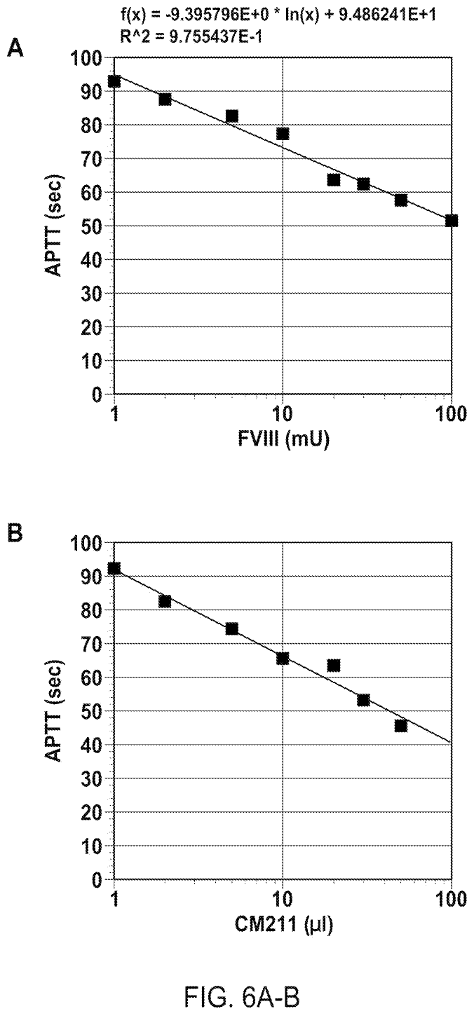

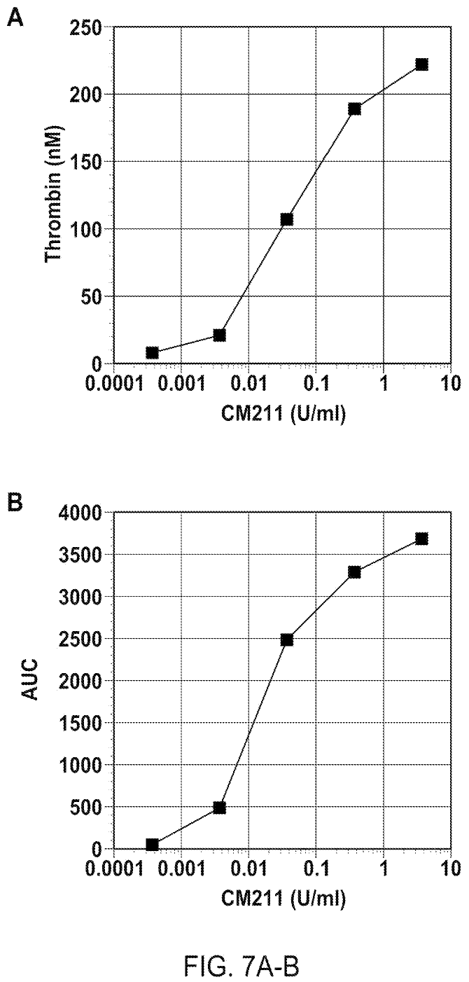

Human albumin has a half life of over 19 days in humans but only 2 days in mice. Since the intention was to use CM110 to prolong the half life of FVIII, a mouse model was employed that expresses the human neonatal Fc receptor (FcRN) and has knocked out production of mouse albumin. The specific mice are B6.CgAlb^{em12mvw}Fcgrt^{tm1Dcr}Tg(FCGRT)32Dcr/Mvwj. These mice recapitulate the appropriate half life of albumin in humans. When a 10 mg/ml solution of CM110 was injected intravenously into the Tg32 mice and decay monitored over the next month, it was found that CM110 had a half life of 92 hrs (FIG. 8A). Human FVIII has a half life of about 4 hrs in these mice.

These same mice to measure the half life of CM211. Twenty APTT units of CM211 were injected intravenously into these mice to measure the half life. Blood was drawn at the indicated time points and CM211 was measured using a human FVIII immunoactivity assay. FIG. 8B shows data from five separate mice. CM211 has a half life of about 55 hours in these mice (FIG. 8B).

Since FcRN is also known to be involved in providing bioavailability of subcutaneous proteins, CM211 was tested by injecting 20 APTT units subcutaneously in the same mice. Factor VIII activity was easily measured in the blood of mice using the Coatest assay, peaking between 8 and 24 hours after injection (FIG. 8C).

### Example 7. Synthesis and Production of CM110short

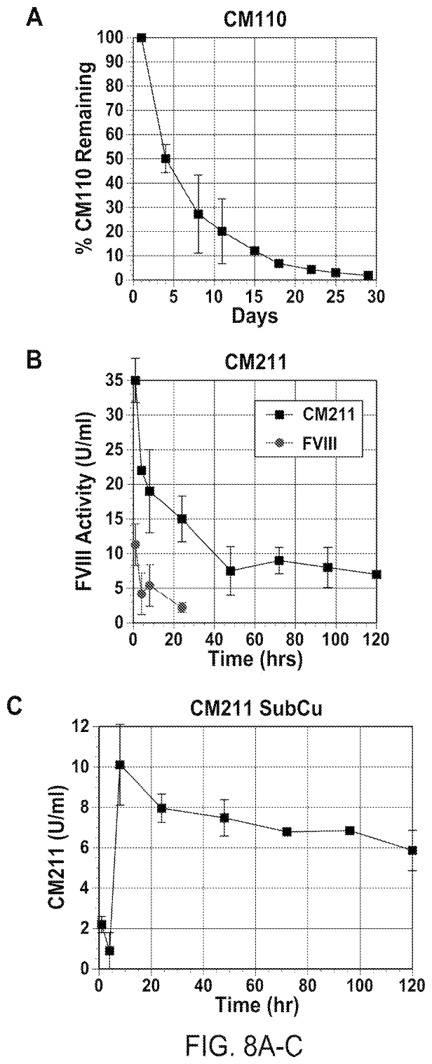

The synthetic protein CM110short was designed to protect FVIII from degradation and nonspecific binding, divorce it from the endogenous vWF and extend its half life in the blood. In specific embodiments, the protein consists of the D′ region of vWF, or D′ and a fragment of D3, a 56 amino acid glycine, serine rich linker, and a full length human albumin. A codon optimized DNA sequence encoding this protein was synthesized by Gene Art and inserted into the expression plasmid pcDNA3.4TOPO. Transient transfection of this plasmid into the human embryonic kidney cell line Expi293 produced substantial quantities of protein in the cell supernate over four days of culture (FIG. 9A). The protein was purified directly from the supernate using affinity chromatography on an HSA affinity column (FIG. 9A). The protein has a predicted molecular weight of 85,310 and was seen to run at about 85,000 on an SDS polyacrylamide gel (FIG. 9A). When the protein was passed over a calibrated Superdex S-200 gel filtration column the protein exhibited a molecular weight of 85,000, suggesting that in solution, it behaves as a monomer (FIG. 9B).

Von Willebrand's factor naturally forms a highly ordered polymer in the Weibel Palade bodies and when secreted travels the blood as a selection of multimers. The D′ fragment has no unpaired cysteines and so does not form these multimers.

### Example 8. CM110short is Cleaved by Thrombin

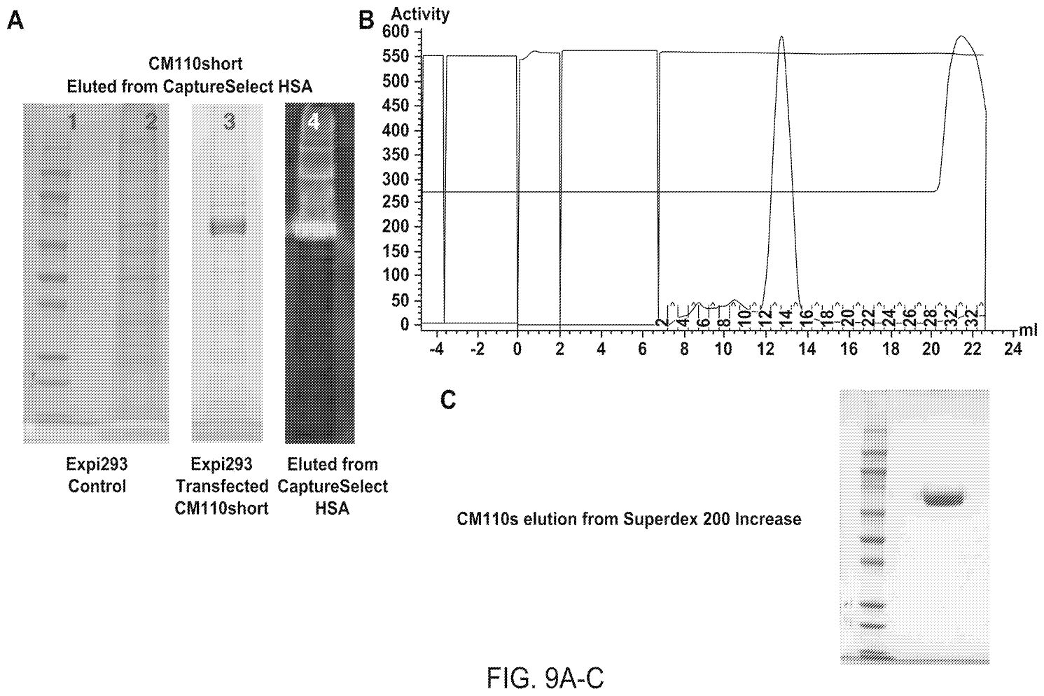

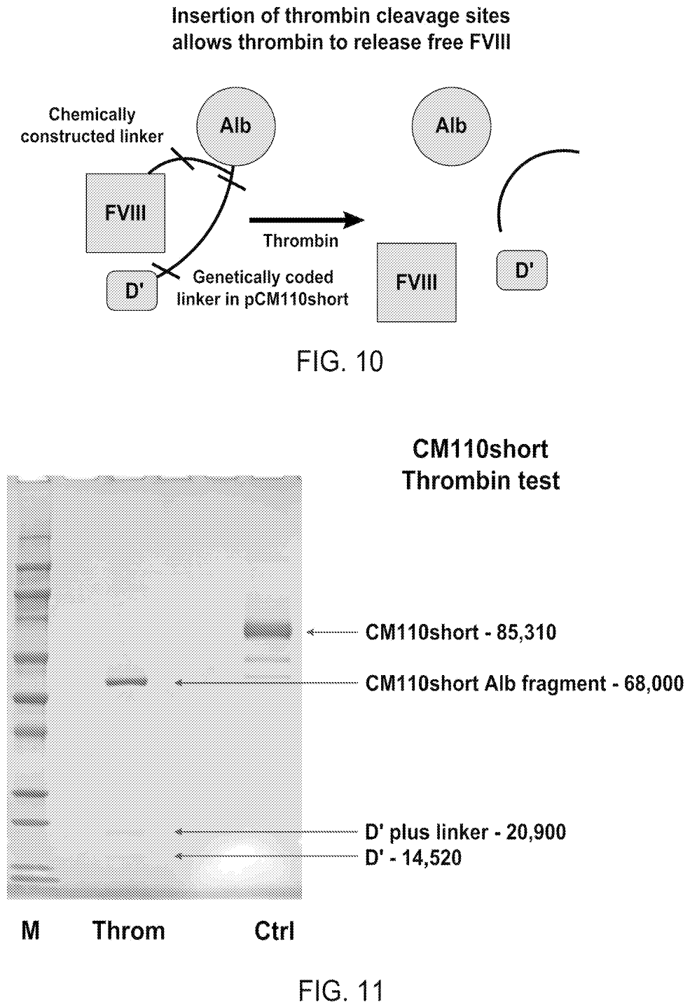

Thrombin activates FVIII to FVIIIa, allowing it to bind FIXa and FX. CM211s has been designed to release free FVIII in response to thrombin generation, as shown in FIG. 10. Two thrombin cleavage sites have been built into the amino acid linker between D′ and albumin in CM110s such that on activation by thrombin, the vWF fragment is fully released from FVIII. FIG. 11 shows that CM110short can be cleaved into free albumin, the D′ plus linker and free D′ by treatment with thrombin.

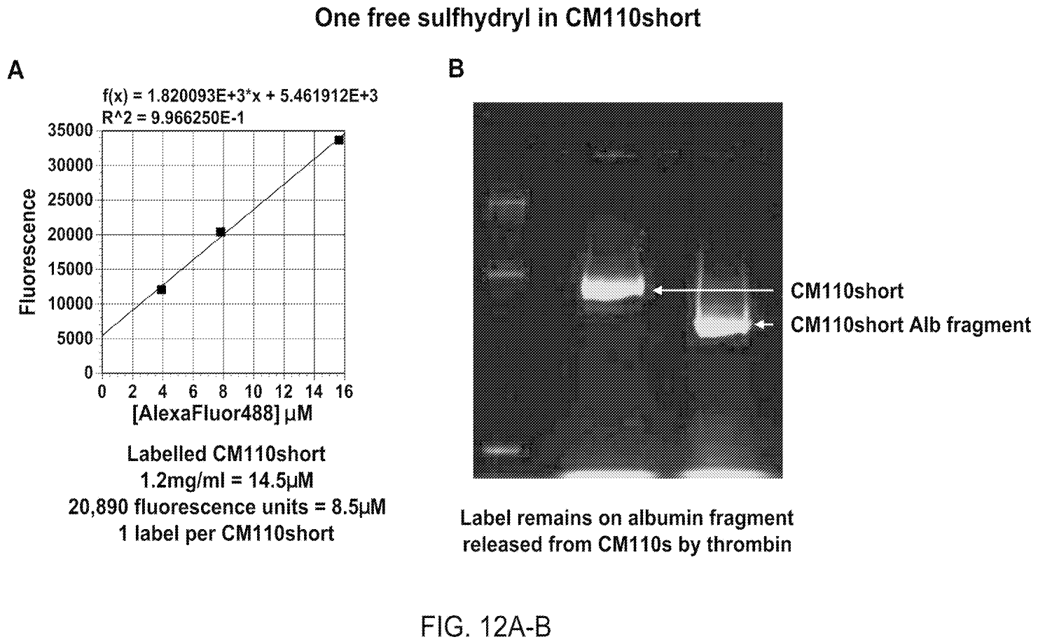

Thrombin treatment of CM110short can also demonstrate another important property of the molecule. In panel A of FIG. 12, CM110short was treated with 1 mM fluoresceinyl maleimide for one hour. Comparison with a standard curve shows that only one molecule of fluorescein is incorporated per molecule of CM110short. Panel B of FIG. 12 demonstrates that after treatment with thrombin, all of the fluorescence stays with the albumin fragment. These results show that there is only a single free sulfhydryl in CM110short and it is cysteine224, corresponding to cys34 in albumin.

### Example 9—Incorporation of a Cleavable Peptide into the Click Chemistry Linker and Formation of CM211s

Disclosed herein is a method of producing a secondary protein and utilizing the natural ability of FVIII to bind to the D′ region of vWF. In this way, the two proteins form the appropriate complex that is then chemically crosslinked. An excess of the D′ containing reagent can be used in order to drive as much of the FVIII into the complex as possible. In such a situation, bifunctional crosslinkers often form polymers, reducing the yield of functional complex. Click chemistry agents are designed to react only with one another, precluding polymer formation. The reaction pair of trans-cyclooctene and methyl tetrazine has the appropriate characteristics of fast, quantitative reactivity.

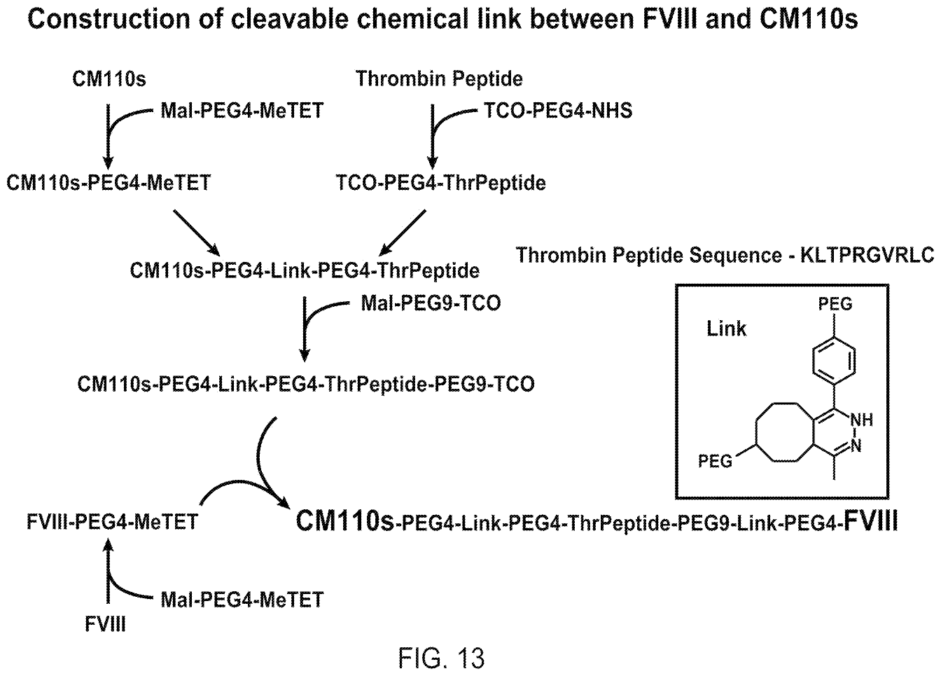

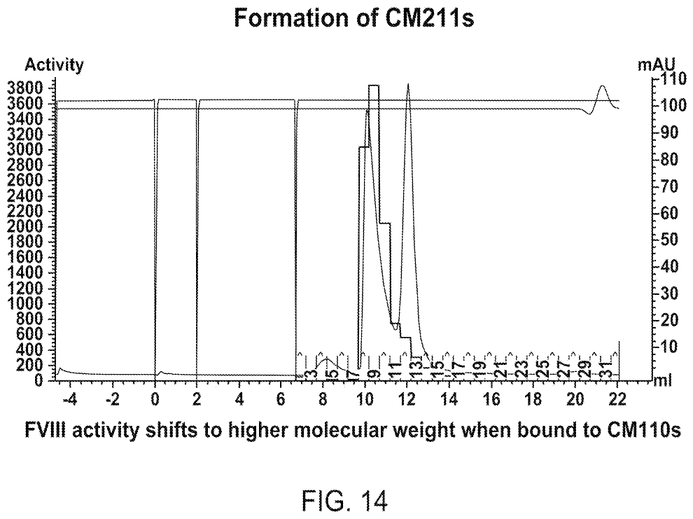

It can be desirable that the overall regulation of blood coagulation not be disturbed by introducing a FVIII that has different reactive properties than the natural protein. For example, if the albumin remained attached to FVIII by a chemical linkage after activation, it could potentially disturb the natural decay of FVIII activity and promote further blood coagulation. To eliminate the potential for this problem, a thrombin cleavable peptide can be introduced into the click chemistry linker. FIG. 13 shows a scheme for incorporating a thrombin cleavage site into the linker. This peptide can be treated with N-hydroxysuccinimide-PEG9-TCO to create a TCO labeled peptide. This can be mixed with CM110short (or CM110) that had previously been labeled with maleimide-PEG4-methyltetrazine. The reaction of the TCO and the methyltetrazine creates a CM110short molecule that now contained the peptide with a terminal cysteine. This molecule can be treated with maleimide-PEG4-methyltetrazine to create CM110short labeled with a peptide containing a terminal methyltetrazine. This molecule can then be mixed with FVIII previously labeled with maleimide-PEG9-TCO. The above process can also be used with CM110 in place of CM110short (Collectively CM110s). The reaction of the click chemistry pair created CM211s (both CM211 and CM211short). FIG. 14 shows that all of the FVIII activity elutes much earlier when chromatographed on Superdex 200 Increase, indicating successful formation of CM211s. Similarly to the thrombin cleavage sites in CM110 and CM110short, the cleavable peptide could also be constructed with a factor Xa cleavage site.

### Example 10. Genetic Fusion of a Modified FVIII and a D′short Containing Fusion Protein

The nucleotide sequence coding for FVIII can be modified to add the coding sequence for the immunoglobulin Fc region. A second gene can be created that encodes the D′D3short, e.g. the D′, fragment of vWF linked to a second immunoglobulin Fc region. These two genes can be inserted into separate plasmids or into a single bicistronic plasmid. When these two sequences are transcribed and translated into protein the cell links them together via disulfide bonds. Such a fusion can be created by substituting D′D3short, e.g. D′, for D′D3 in, for example, the methods disclosed in US Patent Application Publication No. US20150023959.

### Example 11. Use of a FVIII with Inserted Cysteines

Factor VIII can be modified to insert new cysteines for use in attaching a CM110s (Radtke, et al. J. Thromb. Haem. 5, 102-108, (2007), Mei, et al., Blood, 116, 270-279, (2010)). For example, mutation of lysine 1084 to a cysteine places a new link on the surface of the molecule and there is no effect on activity. This mutation has been used to attach a 60 KD PEG molecule to FVIII. Similarly, the new cysteine can be used to attach a click chemistry target, such as maleimide-PEG9-transcyclooctene. This could be then be reacted with a CM110 that had been modified with maleimide-PEG4-methyltetrazine. Reaction of the click pair would synthesize a CM211s using a link other than cys310 or cys692.

### Example 12. Site Specific Addition of a CM110s to Carbohydrate Side Chains of FVIII

FVIII has multiple glycosylation sites, both 0 and N linked (Orlova, 2013). These glycosylation sites can be used as attachment sites for click chemistry based carbohydrates and hence used as anchors for CM110s. For example, N8 is an engineered FVIII that contains only a small fragment of the B domain and retains only a single O-glycosylation site (Thim, 2010). Using a series of enzymes specific for O-glycans, Stennicke, et al. (Stennicke, 2013)) were able to attach a large PEG molecule specifically to that site. A similar approach can be used to insert a click chemistry enabled glycan (Zhang, 2013). After first desialylating the FVIII, azido sialic acid can be specifically added to the single O-glycan site using ST3GalI. The azide can then be targeted by its click chemistry partners BCN (bicyclo octyne) or DBCO (dibenzyl cyclooctene).

### Example 13. Synthesis and Production of CM115

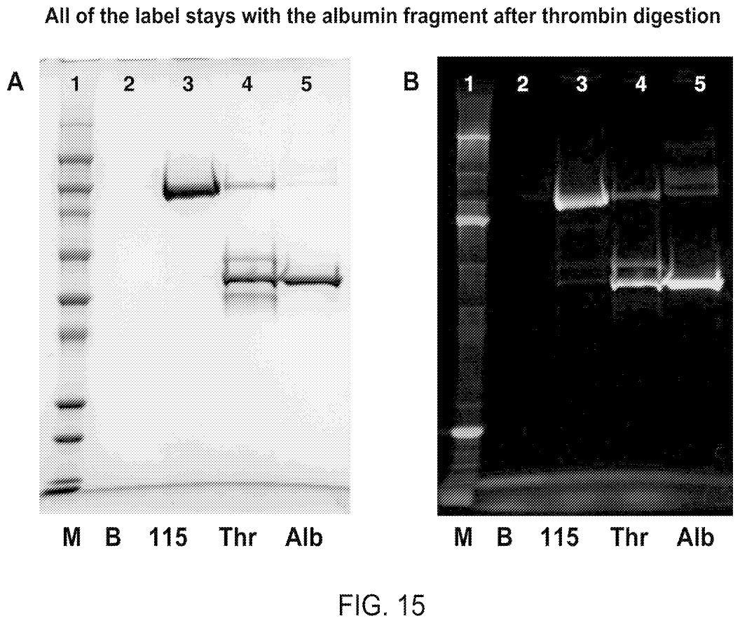

The synthetic protein CM115 (represented by SEQ ID NO: 11) is designed to protect FVIII from degradation and nonspecific binding, divorce it from the endogenous vWF, and extend its half life in the blood. In a specific embodiment, the CM115 protein can consist of a specific D′ D3 short region of vWF, a 68 amino acid glycine, serine-rich linker, and a full length human albumin A codon optimized DNA sequence encoding this protein was synthesized by Gene Art and inserted into the expression plasmid pcDNA3.4TOPO. Transient transfection of this plasmid into the human embryonic kidney cell line Expi293 produced substantial quantities of protein in the cell supernate over four days of culture. The protein was purified directly from the supernate using affinity chromatography on an HSA affinity column. The protein has a predicted molecular weight of 122,714 and was seen to run at about 110,000 on an SDS polyacrylamide gel (FIG. 15A). When CM115 is treated with thrombin, several fragments are created, separating the von Willebrand fragment from albumin. When CM115 is labeled with maleimide—Alexa488 and treated with thrombin, all of the label remains with the albumin fragment (FIG. 15B), demonstrating that only cysteine 34 of albumin is labeled.

### Example 14. Conjugation of CM115 to Factor VIII

CM115, produced as above, was treated with 100 μM maleimide-PEG4-methyltetrazine for one hour. Unreacted maleimide-PEG4-methyltetrazine was removed by size exclusion chromatography using a 40 kd molecular weight cutoff spin column. This labeled CM115 was then reacted with 1.5 mM TCO-labeled cleavable peptide. Unreacted peptide was removed with a similar spin column. The CM115-peptide was then treated with 100 μM maleimide-PEG4-methyltetrazine and again, the unreacted label was removed using the spin column.

This labeled CM115-peptide can then be reacted with a TCO labeled, cysteine modified FVIII. The CM115-FVIII conjugate can then be purified as described above using size exclusion chromatography and a buffer containing 0.25M calcium chloride.

### Materials and Methods

FVIII (B region deleted) was purchased from American Pharma Wholesale.
FVIII activity assay—FVIII activity was measured using the Coamatic FVIII chromogenic assay (Diapharma).
Gel electrophoresis—Samples were run on 4-12% Bis Tris Plus gradient gels (ThermoFisher).
Thrombin generation assay—Thrombin generation was measured using the fluorogenic Technothrombin Thrombin Generation kit and reagents from Diapharma, measured on a BioTek FL-600 plate reader. For thrombin generation using CM211, known concentrations of the protein were diluted into Technoclone FVIII-deficient plasma. Each assay also contained Technoclone Technothrombin TGA substrate, and Technoclone low RC. All reagents from Diapharma.
Activated Partial Thromboplastin Time—Activated partial thromboplastin time was measured using Technoclone Siron LIS liquid (Diapharma) on a Labomed SCO-2000 coagulometer.
Generation and purification of CM110-CM110, the companion protein, consists of the D′D3 region of human von Willebrand Factor, a linker (for example 56 amino acid glycine serine rich amino acid linker), and a full length human albumin Plasmid pCM110 contains amino acids 1-22 and 764-1247 of the human von Willebrand factor, a 56 amino acid linker, amino acids 25-609 of the human albumin and a 6×his tag. Plasmid pCM110RM contains alanines substituted for the cysteines corresponding to cys1099 and cys1142 in vWF. Plasmid pCM110RMHM has the alanine mutations but has removed the His tag. The albumin affinity column was much more efficient that HisTRAP purification, so all work described uses plasmid pCM110RMHM.
Generation and purification of CM110short-CM110short, the companion protein, consists of the D′D3short region of human von Willebrand Factor (for example, serine764 through cysteine 1031, serine 764 through asparagine 864 (the full D′ domain), serine 764 through cysteine 863, leucine 765 through cysteine 863, leucine 765 through asparagine 864, serine 766 through cysteine 863, serine 766 through asparagine 864, serine 764 though arginine 1035, serine 764 though lysine 1036, serine 764 through serine 900, serine 764 through cysteine 1099, serine 764 through cysteine 1142, and serine through proline 1240); a linker (for example 56 amino acid glycine serine rich amino acid linker); and a full length human albumin, an albumin fragment, an immunoglobulin Fc domain, or an Fc fragment. One exemplary CM110short (CM110short764-863LWA) comprises amino acids 1-22 and 764-863 of the human von Willebrand factor, a 56 amino acid linker, and a full length human albumin. Plasmid pCM110short contains amino acids 1-22 and 764-863 of the human von Willebrand factor, a 56 amino acid linker, amino acids 25-609 of the human albumin and a 6×his tag. Plasmid pCM110shortHM has removed the His tag. The albumin affinity column was much more efficient that HisTRAP purification, so all work described uses plasmid pCM110HM.

Generation and purification of CM115-CM115, a companion protein, consists of the D′ and a specific portion of the D3 region of human von Willebrand Factor (serine 764 through proline 1197), a linker (for example a 68 amino acid glycine serine rich linker containing two thrombin recognition sites) and a full length albumin, an albumin fragment, an immunoglobulin Fc domain or an Fc fragment. One exemplary CM115 (CM115EM) comprises amino acids 1-22 and 764-1197 of the human von Willebrand Factor, a 68 amino acid linker and a full length human albumin (SEQ ID NO: 11). Plasmid CM115EM contains amino acids 1-22, 764-1197 of the human von Willebrand Factor, a 68 amino acid linker and amino acids 25-609 of the full-length albumin.

Each of the constructs were codon optimized, synthesized and inserted into pcDNA3.4TOPO by Gene Art (ThermoFisher). Plasmids were transfected into E. coli One Shot Mach1 T1 competent bacteria (ThermoFisher) and purified using PureLink Hipure plasmid filter kits. Purified plasmid was transfected into Expi293 HEK cells (ThermoFisher) using the Expifectamine293 transfection kit, according to the manufacturer's instructions (ThermoFisher).

Four days after transfection, the medium was harvested and cells and cellular debris were removed by centrifugation at 7,500×g for 20 minutes. Clarified supernate (500 ml) was applied directly to a POROS CaptureSelect HSA 10×100 mm column (ThermoFisher) equilibrated with 20 mM HEPES, pH 7.4, 150 mM NaCl at a flow rate of 3 ml/min using an Akta Pure chromatography system (GE Lifesciences) equipped with a 50 ml Superloop. After the entire sample had been applied, the column was washed with a further 10 column volumes of the same buffer. CM110 or CM110short was eluted from the column using the same buffer containing 2M MgCl2. CM110 or CM110short was then passed over Zeba 10 ml spin desalting columns (ThermoFisher) to equilibrate into 20 mM HEPES, pH 7.4, 150 mM NaCl, 4 mM CaCl2. We routinely recover about 15 mg of CM110 from 500 ml of culture fluid. CM110 is recovered in similar quantities. CM110 or CM110short is quantitated using the SimpleStep HSA Elisa kit (Abcam) combined with protein measurement. CM110 is 55% albumin by weight. CM110 short shown in Example 7 is 80% albumin by weight. CM115 is 55% albumin by weight.

Click chemistry—Methyltetrazine-PEG4-maleimide (Tet-P4-mal) and trans-cyclooctene-PEG12-N-hydroxysuccinimide (TCO-P12-NHS) were obtained from Broadpharm, dissolved in dimethylsulfoxide at 10 mM and stored in liquid nitrogen.
CM110 (or CM110short), 0.5 ml at 5 mg/ml in 20 mM HEPES, pH 7.4, 150 mM NaCl, 4 mM CaCl2, was treated with 1 mM TCO-P12-NHS for two hours at room temperature in the dark. The solution was then applied to a calibrated Superdex S-200 Increase column (10×300 mm) equilibrated with 20 mM HEPES, pH 7.4, 300 mM NaCl, 4 mM CaCl2. Fractions containing CM110 (or CM110short) were identified by A280 and pooled.
B region deleted factor VIII (FVIII) was obtained from American Pharma Wholesale. Approximately 6,000 IU were dissolved in 1 ml of water directly from three 2,000 IU vials. Tet-P4-mal was added to 0.1 mM and the solution was incubated for 2 hrs at room temperature in the dark. Aliquots of 0.5 ml were passed over a Superdex S-200 Increase column (10×300) equilibrated with 20 mM HEPES, pH 7.4, 300 mM NaCl, 4 mM CaCl2, fractions collected by A280 and assayed for FVIII activity. Factor VIII activity was measured using the Coamatic FVIII assay (Diapharma). Fractions containing activity were pooled.

The labelled CM110 (or CM110short) and FVIII were combined, diluted 1 to 2 with water to reduce the NaCl to 150 mM and concentrated to 0.2 ml using Amicon Ultra 15 centrifugal filters (Ultaracel—30K, Millipore). The filter was rinsed with 0.1 ml 20 mM HEPES, pH 7.4, 150 mM NaCl, 4 mM CaCl₂) which was added to the original solution. The solution was incubated at room temperature for 2 hours in the dark.

The solution was applied to a Superdex S-200 Increase column (10×300) equilibrated with 20 mM HEPES, pH 7.4, 300 mM NaCl, 4 mM CaCl₂). Fractions containing FVIII activity were pooled and frozen in liquid nitrogen.

Measurement of label incorporation—FVIII label incorporation was measured by incubating a known concentration by IU of FVIII, exactly as described above but using fluoresceinyl maleimide (SigmaAldrich). CM110 or CM110short labeling was estimated by labeling as above then reacting with 1 mM bodipy FL—tetrazine. In each case, after labeling, the solution was passed over a small Zeba spin column to remove free label. Fluorescence was read on a BioTek FL-600.

Fluorescently labeled FVIII was incubated with 1 U of thrombin (SigmaAldrich) for 10 minutes at 37° C. Gel electrophoresis buffer was added to stop the reaction and the sample was run on a 4-12% Bis Tris gel.

Mouse studies—All mouse experiments were carried out at Jackson Laboratories. Half life and functional studies were carried out by injecting 0.2 ml of the appropriate protein solution either intravenously or subcutaneously. For FVIII functional and half life studies, the solution contained 100 IU/ml, as measured via Coatest assay. For CM110 half-life measurement, the solution contained 10 mg/ml. For CM211, the solution contained 100 U as measured by APTT, which correspond to about 600 U/ml via Coatest.

### REFERENCES

Andersen, J. T., Pehrson, R., Tolmachev, V., Daba, M. B., Abrahmsén, L., & Ekblad, C. (2011). Extending half-life by indirect targeting of the neonatal Fc receptor (FcRn) using a minimal albumin binding domain. The Journal of Biological Chemistry, 286(7), 5234-5241. Retrieved from
Bradbury, A. R. M., Sidhu, S., Dübel, S., & McCafferty, J. (2011). Beyond natural antibodies: the power of in vitro display technologies. Nature Publishing Group, 29(3), 245-254.
Buyue, Y., Liu, T., Kulman, J. D., Toby, G. G., Kamphaus, G. D., Patarroyo-White, S., et al. (2014). A Single Chain Variant of Factor VIII Fc Fusion Protein Retains Normal In Vivo Efficacy but Exhibits Altered In Vitro Activity. PLoS ONE, 9(11), e113600.
Ginn, C., Khalili, H., Lever, R., & Brocchini, S. (2014). PEGylation and its impact on the design of new protein-based medicines. Future Medicinal Chemistry, 6(16), 1829-1846.
Hoots, W. K. (2003). Comprehensive care for hemophilia and related inherited bleeding disorders: why it matters. Current Hematology Reports, 2(5), 395-401.
Horisawa, K. (2014). Specific and quantitative labeling of biomolecules using click chemistry. Frontiers in Physiology 5, 457.
Kelly, J. H., & Sussman, N. L. (2000). A Fluorescent Cell-Based Assay for Cytochrome P-450 Isozyme 1A2 Induction and Inhibition. Journal of Biomolecular Screening, 5(4), 249-253.
Kempton, C. L., & Meeks, S. L. (2014). Toward optimal therapy for inhibitors in hemophilia. Hematology/the Education Program of the American Society of Hematology. American Society of Hematology. Education Program, 2014(1), 364-371.
Kramer, R. H., & Karpen, J. W. (1998). Spanning binding sites on allosteric proteins with polymer-linked ligand dimers. Nature, 395(6703), 710-713.
Lawson, J. H., Butenas, S., Ribarik, N., & Mann, K. G. (1993). Complex-dependent inhibition of factor VIIa by antithrombin III and heparin. The Journal of Biological Chemistry, 268(2), 767-770.
Lenting, P. J., Christophe, O. D., & Denis, C. V. (2015). von Willebrand factor biosynthesis, secretion, and clearance: connecting the far ends. Blood, 125(13), 2019-2028.
Mannucci, P. M., & Mancuso, M. E. (2014). Fc-fusion technology and recombinant FVIII and FIX in the management of the hemophilias. Drug Design, Development and Therapy, 365.
Mei, B., Pan, C., Jiang, H., Tjandra, H., Strauss, J., Chen, Y., et al. (2010). Rational design of a fully active, long-acting PEGylated factor VIII for hemophilia A treatment. Blood, 116(2), 270-279.
Oldenburg, J., & Albert, T. (2014). Novel products for haemostasis—current status. Haemophilia, 20,23-28.
Orlova, N. A., Kovnir, S. V., Vorobiev, I. I., Gabibov, A. G., & Vorobiev, A. I. (2013). Blood Clotting Factor VIII: From Evolution to Therapy. Acta Naturae, 5(2), 19-39.
Pasut, G., & Veronese, F. M. (2012). State of the art in PEGylation: The great versatility achieved after forty years of research. Journal of Controlled Release, 161(2), 461-472.
Peters, T., Jr. (1995). ALL ABOUT ALBUMIN: BIOCHEMISTRY, GENETICS, AND MEDICAL APPLICATIONS. Academic Press, New York, N.Y. 1996.
Philips, J.-C., & Scheen, A. (2006). Insulin detemir in the treatment of type 1 and type 2 diabetes. Vascular Health and Risk Management, 2(3), 277-283.
Pipe, S. W. (2010). Hemophilia: new protein therapeutics. Hematology/the Education Program of the American Society of Hematology. American Society of Hematology. Education Program, 2010,203-209.
Powell, J S. (2014) Lasting power of new clotting proteins. ASH Education Book 2014,355-363.
Radtke, K P, Griffin, J H, Riceberg, J., Gale, A J (2007) Disulfide bond-stabilized factor VIII has prolonged factor VIIIa activity and improved potency in whole blood clotting assays. J. Thromb. Heam. 5,102-108.
Ran, F. A., Hsu, P. D., Lin, C.-Y., Gootenberg, J. S., Konermann, S., Trevino, A. E., et al. (2013). Double Nicking by RNA-Guided CRISPR Cas9 for Enhanced Genome Editing Specificity. Cell, 1-15.
Schulte, S. (2008). Use of albumin fusion technology to prolong the half-life of recombinant factor VIIa. Thrombosis Research, 122(S4), S14-S19.
Smith, S. A., Travers, R. J., & Morrissey, J. H. (2015). How it all starts: Initiation of the clotting cascade. Critical Reviews in Biochemistry and Molecular Biology, 1-11.
Srivastava, A., Brewer, A. K., Mauser-Bunschoten, E. P., Key, N. S., Kitchen, S., Llinas, A., et al. (2012). Guidelines for the management of hemophilia. Haemophilia, 19(1), e1-e47.
Stennicke, H. R., Kjalke, M., Karpf, D. M., Balling, K. W., Johansen, P. B., Elm, T., et al. (2013). A novel B-domain 0-glycoPEGylated FVIII (N8-GP) demonstrates full efficacy and prolonged effect in hemophilic mice models. Blood, 121(11), 2108-2116.
Thim, L, et al. (2010) Purification and characterization of a new recombinant factor VIII (N8). Haemophilia 16,349-359.
Vadivel, K., & Bajaj, S. P. (2012). Structural biology of factor VIIa/tissue factor initiated coagulation. Frontiers in Bioscience: a Journal and Virtual Library, 17,2476-2494.
van der Flier, A., Liu, Z., Tan, S., Chen, K., Drager, D., Liu, T., et al. (2015). FcRn Rescues Recombinant Factor VIII Fc Fusion Protein from a VWF Independent FVIII Clearance Pathway in Mouse Hepatocytes. PLoS ONE, 10(4), e0124930-23.
Wakabayashi, H., & Fay, P. J. (2013). Molecular orientation of Factor VIIIa on the phospholipid membrane surface determined by fluorescence resonance energy transfer. The Biochemical Journal, 452(2), 293-301. http://doi.org/10.1042/BJ20130025
Wakabayashi, H., Koszelak, M. E., Mastri, M., & Fay, P. J. (2001). Metal Ion-independent Association of Factor VIII Subunits and the Roles of Calcium and Copper Ions for Cofactor Activity and Inter-Subunit Affinity t. Biochemistry, 40(34), 10293-10300. http://doi.org/10.1021/bi010353q
Yee, A., Gildersleeve, R. D., Gu, S., Kretz, C. A., McGee, B. M., Carr, K. M., et al. (2014). A von Willebrand factor fragment containing the D′D3 domains is sufficient to stabilize coagulation factor VIII in mice. Blood, 124(3), 445-452.
Zhang, X., Zhang, Y. (2013) Applications of azide based bioorthogonal click chemistry in glycobiology. Molecules 18,7145-7159.
Zhou, H.-X. (2006). Quantitative Relation between Intermolecular and Intramolecular Binding of Pro-Rich Peptides to SH3 Domains. Biophysical Journal, 91(9), 3170-3181.

SEQ ID NO: 1:
CM110RM-The sequence of CM110 with the
two cysteines changed to alanines
ATGATCCCCGCCAGATTCGCCGGCGTGCTGCTGGC
CCTGGCCCTGATCCTGCCTGGCACCCTGTGTAGCC
TGAGCTGCAGACCCCCCATGGTCAAGCTCGTGTGC
CCTGCCGACAACCTGCGGGCCGAGGGCCTGGAATG
CACCAAGACCTGCCAGAACTACGATCTGGAATGCA
TGAGCATGGGCTGCGTGTCCGGCTGCCTGTGCCCT
CCTGGAATGGTCCGACACGAGAACAGATGCGTGGC
CCTGGAACGGTGCCCATGCTTTCATCAAGGCAAAG
AGTACGCCCCTGGCGAGACAGTGAAGATCGGCTGC
AATACCTGCGTGTGCCGGGACCGGAAGTGGAACTG
CACCGACCACGTGTGCGACGCCACCTGTAGCACCA
TCGGCATGGCCCACTACCTGACCTTCGACGGCCTG
AAGTACCTGTTCCCCGGCGAGTGCCAGTACGTGCT
GGTGCAGGACTACTGCGGCAGCAACCCCGGCACCT
TCCGGATCCTCGTGGGCAACAAGGGCTGCAGCCAC
CCCAGCGTGAAGTGCAAGAAGCGGGTCACCATCCT
GGTGGAAGGCGGCGAGATCGAGCTGTTCGACGGCG
AAGTGAACGTGAAGCGGCCCATGAAGGACGAGACA
CACTTCGAGGTGGTGGAAAGCGGCCGGTACATCAT
CCTGCTGCTGGGCAAGGCCCTGAGCGTCGTGTGGG
ACCGGCACCTGAGCATCAGCGTGGTGCTGAAGCAG
ACCTACCAGGAAAAAGTCTGCGGCCTCTGCGGCAA
CTTCGACGGCATCCAGAACAACGACCTGACCAGCA
GCAACCTGCAGGTCGAAGAGGACCCCGTGGACTTC
GGCAACAGCTGGAAGGTGTCCAGCCAGTGCGCCGA
CACCAGAAAGGTGCCCCTGGACAGCAGCCCCGCCA
CCTGTCACAACAACATCATGAAGCAGACAATGGTG
GACAGCTCCTGCCGGATCCTGACCAGCGACGTGTT
CCAGGACTGCAACAAGCTGGTGGACCCCGAGCCCT
ACCTGGACGTGTGCATCTACGATACCTGCAGCTGC
GAGAGCATCGGCGACTGCGCCGCCTTCTGCGACAC
AATCGCCGCCTACGCCCATGTGTGCGCCCAGCACG
GCAAGGTGGTCACCTGGCGGACCGCCACCCTGTGC
CCACAGAGCTGCGAGGAACGGAACCTGCGCGAGAA
CGGCTACGAGGCCGAGTGGCGGTACAACAGCTGCG
CCCCTGCCTGCCAGGTCACATGCCAGCACCCTGAG
CCTCTGGCCTGCCCCGTGCAGTGCGTGGAAGGCTG
TCACGCCCACTGCCCTCCCGGCAAGATCCTGGACG
AGCTGCTGCAGACCTGCGTGGACCCTGAGGACTGC
CCTGTGTGCGAGGTGGCCGGCAGAAGATTCGCCAG
CGGCAAGAAAGTGACCCTGAACCCCAGCGACCCCG
AGCACTGCCAGATCTGCCACTGCGACGTGGTCAAT
CTGACCTGCGAGGCCTGTCAGGAACCTGGCGGCCT
GGTCGTGCCTCCTGGCGGAAGAGGTGGCGGAGGCT
CTGGGGGAGGTTCTGGCGGAGGAAGCGAGGGCGGA
GGATCTGAGGGTGGCGGCTCTGAAGGTGGCGGAAG
CGAAGGGGGAGGCTCCGAAGGCGGTGGATCTGAAG
GGGGGGGATCTGGCGGCGGATCTGGAAGCGGTGGA
AGAGGCGACGCCCACAAGTCCGAGGTGGCCCACAG
ATTCAAGGACCTGGGCGAGGAAAACTTCAAGGCCC
TGGTGCTGATCGCCTTCGCCCAGTACCTGCAGCAG
TGCCCCTTCGAGGACCACGTGAAGCTGGTCAACGA
AGTGACCGAGTTCGCCAAGACCTGTGTGGCCGACG
AGAGCGCCGAGAACTGCGACAAGAGCCTGCACACC
CTGTTCGGCGACAAGCTGTGCACCGTGGCCACCCT
GCGGGAAACCTACGGCGAGATGGCCGACTGCTGCG
CCAAGCAGGAACCCGAGCGGAACGAGTGCTTCCTG
CAGCACAAGGACGACAACCCCAACCTGCCCAGACT
CGTGCGGCCCGAGGTGGACGTGATGTGCACCGCCT
TCCACGACAACGAGGAAACCTTCCTGAAGAAGTAC
CTCTACGAGATCGCCAGACGGCACCCCTACTTCTA
CGCCCCCGAGCTGCTGTTCTTCGCCAAGCGGTACA
AGGCCGCCTTCACCGAGTGCTGCCAGGCCGCCGAT
AAGGCCGCCTGCCTGCTGCCCAAGCTGGATGAGCT
GAGGGACGAGGGCAAGGCCAGCTCCGCCAAGCAGA
GACTGAAGTGCGCCAGCCTGCAGAAGTTCGGCGAG
CGGGCCTTTAAGGCCTGGGCCGTGGCCCGGCTGAG
CCAGAGATTCCCCAAGGCCGAGTTTGCCGAGGTGT
CCAAGCTGGTCACCGATCTGACCAAGGTGCACACC
GAGTGTTGTCACGGCGACCTGCTGGAATGCGCCGA
CGACAGAGCCGACCTGGCCAAGTACATCTGCGAGA
ACCAGGACAGCATCAGCTCCAAGCTGAAAGAGTGC
TGCGAGAAGCCCCTGCTGGAAAAGAGCCACTGTAT
CGCCGAGGTGGAAAACGACGAGATGCCCGCCGACC
TGCCCAGCCTGGCCGCCGACTTCGTGGAAAGCAAG
GATGTGTGCAAGAACTACGCCGAGGCCAAGGACGT
GTTCCTGGGCATGTTCCTGTACGAGTACGCCCGCA
GACACCCCGACTACTCCGTGGTGCTGCTGCTGCGG
CTGGCCAAGACCTACGAGACAACCCTGGAAAAGTG
CTGCGCCGCTGCCGACCCCCACGAGTGCTACGCCA
AGGTGTTCGACGAGTTCAAGCCTCTGGTGGAAGAA
CCCCAGAACCTGATCAAGCAGAACTGCGAGCTGTT
TGAGCAGCTGGGCGAGTACAAGTTCCAGAACGCCC
TGCTCGTGCGGTACACCAAGAAAGTGCCTCAGGTG
TCCACCCCCACACTGGTGGAAGTGTCCCGGAACCT
GGGCAAAGTGGGCTCCAAGTGCTGCAAGCACCCAG
AGGCCAAGCGGATGCCCTGCGCCGAGGACTACCTG
AGCGTGGTCCTGAACCAGCTGTGCGTGCTGCACGA
GAAAACCCCCGTGTCCGACAGAGTGACCAAGTGCT
GTACCGAGAGCCTGGTCAACAGACGGCCCTGCTTC
AGCGCCCTGGAAGTGGACGAGACATACGTGCCCAA
AGAGTTCAACGCCGAGACATTCACCTTCCACGCCG
ACATCTGCACCCTGTCCGAGAAAGAGCGGCAGATC
AAGAAGCAGACCGCCCTGGTCGAGCTGGTCAAGCA
CAAGCCCAAGGCCACCAAAGAACAGCTGAAGGCCG
TGATGGACGACTTCGCCGCCTTCGTCGAGAAGTGT
TGCAAGGCCGACGACAAAGAGACATGCTTCGCCGA
AGAGGGCAAGAAACTGGTGGCCGCCTCTCAGGCCG
CCCTGGGCCTGCACCACCACCACCACCAC
SEQ ID NO: 2:
CM110RMHM-CM110 with the cysteines
changed to alanines and without the
6XHis tag
ATGAGCCTGAGCTGCAGACCCCCCATGGTCAAGCT
CGTGTGCCCTGCCGACAACCTGCGGGCCGAGGGCC
TGGAATGCACCAAGACCTGCCAGAACTACGATCTG
GAATGCATGAGCATGGGCTGCGTGTCCGGCTGCCT
GTGCCCTCCTGGAATGGTCCGACACGAGAACAGAT
GCGTGGCCCTGGAACGGTGCCCATGCTTTCATCAA
GGCAAAGAGTACGCCCCTGGCGAGACAGTGAAGAT
CGGCTGCAATACCTGCGTGTGCCGGGACCGGAAGT
GGAACTGCACCGACCACGTGTGCGACGCCACCTGT
AGCACCATCGGCATGGCCCACTACCTGACCTTCGA
CGGCCTGAAGTACCTGTTCCCCGGCGAGTGCCAGT
ACGTGCTGGTGCAGGACTACTGCGGCAGCAACCCC
GGCACCTTCCGGATCCTCGTGGGCAACAAGGGCTG
CAGCCACCCCAGCGTGAAGTGCAAGAAGCGGGTCA
CCATCCTGGTGGAAGGCGGCGAGATCGAGCTGTTC
GACGGCGAAGTGAACGTGAAGCGGCCCATGAAGGA
CGAGACACACTTCGAGGTGGTGGAAAGCGGCCGGT
ACATCATCCTGCTGCTGGGCAAGGCCCTGAGCGTC
GTGTGGGACCGGCACCTGAGCATCAGCGTGGTGCT
GAAGCAGACCTACCAGGAAAAAGTCTGCGGCCTCT
GCGGCAACTTCGACGGCATCCAGAACAACGACCTG
ACCAGCAGCAACCTGCAGGTCGAAGAGGACCCCGT
GGACTTCGGCAACAGCTGGAAGGTGTCCAGCCAGT
GCGCCGACACCAGAAAGGTGCCCCTGGACAGCAGC
CCCGCCACCTGTCACAACAACATCATGAAGCAGAC
AATGGTGGACAGCTCCTGCCGGATCCTGACCAGCG
ACGTGTTCCAGGACTGCAACAAGCTGGTGGACCCC
GAGCCCTACCTGGACGTGTGCATCTACGATACCTG
CAGCTGCGAGAGCATCGGCGACTGCGCCGCCTTCT
GCGACACAATCGCCGCCTACGCCCATGTGTGCGCC
CAGCACGGCAAGGTGGTCACCTGGCGGACCGCCAC
CCTGTGCCCACAGAGCTGCGAGGAACGGAACCTGC
GCGAGAACGGCTACGAGGCCGAGTGGCGGTACAAC
AGCTGCGCCCCTGCCTGCCAGGTCACATGCCAGCA
CCCTGAGCCTCTGGCCTGCCCCGTGCAGTGCGTGG
AAGGCTGTCACGCCCACTGCCCTCCCGGCAAGATC
CTGGACGAGCTGCTGCAGACCTGCGTGGACCCTGA
GGACTGCCCTGTGTGCGAGGTGGCCGGCAGAAGAT
TCGCCAGCGGCAAGAAAGTGACCCTGAACCCCAGC
GACCCCGAGCACTGCCAGATCTGCCACTGCGACGT
GGTCAATCTGACCTGCGAGGCCTGTCAGGAACCTG
GCGGCCTGGTCGTGCCTCCTGGCGGAAGAGGTGGC
GGAGGCTCTGGGGGAGGTTCTGGCGGAGGAAGCGA
GGGCGGAGGATCTGAGGGTGGCGGCTCTGAAGGTG
GCGGAAGCGAAGGGGGAGGCTCCGAAGGCGGTGGA
TCTGAAGGGGGGGGATCTGGCGGCGGATCTGGAAG
CGGTGGAAGAGGCGACGCCCACAAGTCCGAGGTGG
CCCACAGATTCAAGGACCTGGGCGAGGAAAACTTC
AAGGCCCTGGTGCTGATCGCCTTCGCCCAGTACCT
GCAGCAGTGCCCCTTCGAGGACCACGTGAAGCTGG
TCAACGAAGTGACCGAGTTCGCCAAGACCTGTGTG
GCCGACGAGAGCGCCGAGAACTGCGACAAGAGCCT
GCACACCCTGTTCGGCGACAAGCTGTGCACCGTGG
CCACCCTGCGGGAAACCTACGGCGAGATGGCCGAC
TGCTGCGCCAAGCAGGAACCCGAGCGGAACGAGTG
CTTCCTGCAGCACAAGGACGACAACCCCAACCTGC
CCAGACTCGTGCGGCCCGAGGTGGACGTGATGTGC
ACCGCCTTCCACGACAACGAGGAAACCTTCCTGAA
GAAGTACCTCTACGAGATCGCCAGACGGCACCCCT
ACTTCTACGCCCCCGAGCTGCTGTTCTTCGCCAAG
CGGTACAAGGCCGCCTTCACCGAGTGCTGCCAGGC
CGCCGATAAGGCCGCCTGCCTGCTGCCCAAGCTGG
ATGAGCTGAGGGACGAGGGCAAGGCCAGCTCCGCC
AAGCAGAGACTGAAGTGCGCCAGCCTGCAGAAGTT
CGGCGAGCGGGCCTTTAAGGCCTGGGCCGTGGCCC
GGCTGAGCCAGAGATTCCCCAAGGCCGAGTTTGCC
GAGGTGTCCAAGCTGGTCACCGATCTGACCAAGGT
GCACACCGAGTGTTGTCACGGCGACCTGCTGGAAT
GCGCCGACGACAGAGCCGACCTGGCCAAGTACATC
TGCGAGAACCAGGACAGCATCAGCTCCAAGCTGAA
AGAGTGCTGCGAGAAGCCCCTGCTGGAAAAGAGCC
ACTGTATCGCCGAGGTGGAAAACGACGAGATGCCC
GCCGACCTGCCCAGCCTGGCCGCCGACTTCGTGGA
AAGCAAGGATGTGTGCAAGAACTACGCCGAGGCCA
AGGACGTGTTCCTGGGCATGTTCCTGTACGAGTAC
GCCCGCAGACACCCCGACTACTCCGTGGTGCTGCT
GCTGCGGCTGGCCAAGACCTACGAGACAACCCTGG
AAAAGTGCTGCGCCGCTGCCGACCCCCACGAGTGC
TACGCCAAGGTGTTCGACGAGTTCAAGCCTCTGGT
GGAAGAACCCCAGAACCTGATCAAGCAGAACTGCG
AGCTGTTTGAGCAGCTGGGCGAGTACAAGTTCCAG
AACGCCCTGCTCGTGCGGTACACCAAGAAAGTGCC
TCAGGTGTCCACCCCCACACTGGTGGAAGTGTCCC
GGAACCTGGGCAAAGTGGGCTCCAAGTGCTGCAAG
CACCCAGAGGCCAAGCGGATGCCCTGCGCCGAGGA
CTACCTGAGCGTGGTCCTGAACCAGCTGTGCGTGC
TGCACGAGAAAACCCCCGTGTCCGACAGAGTGACC
AAGTGCTGTACCGAGAGCCTGGTCAACAGACGGCC
CTGCTTCAGCGCCCTGGAAGTGGACGAGACATACG
TGCCCAAAGAGTTCAACGCCGAGACATTCACCTTC
CACGCCGACATCTGCACCCTGTCCGAGAAAGAGCG
GCAGATCAAGAAGCAGACCGCCCTGGTCGAGCTGG
TCAAGCACAAGCCCAAGGCCACCAAAGAACAGCTG
AAGGCCGTGATGGACGACTTCGCCGCCTTCGTCGA
GAAGTGTTGCAAGGCCGACGACAAAGAGACATGCT
TCGCCGAAGAGGGCAAGAAACTGGTGGCCGCCTCT
CAGGCCGCCCTGGGCCTG
SEQ ID NO: 3
amino acid sequence of full D′ D3
sequence defined as ser 764-phe1270
SLSCRPPMVKLVCPADNLRAEGLECTKTCQNYDLE
CMSMGCVSGCLCPPGMVRHENRCVALERCPCFHQG
KEYAPGETVKIGCNTCVCRDRKWNCTDHVCDATCS
TIGMAHYLTFDGLKYLFPGECQYVLVQDYCGSNPG
TFRILVGNKGCSHPSVKCKKRVTILVEGGEIELFD
GEVNVKRPMKDETHFEVVESGRYIILLLGKALSVV
WDRHLSISVVLKQTYQEKVCGLCGNFDGIQNNDLT
SSNLQVEEDPVDFGNSWKVSSQCADTRKVPLDSSP
ATCHNNIMKQTMVDSSCRILTSDVFQDCNKLVDPE
PYLDVCIYDTCSCESIGDCAAFCDTIAAYAHVCAQ
HGKVVTWRTATLCPQSCEERNLRENGYEAEWRYNS
CAPACQVTCQHPEPLACPVQCVEGCHAHCPPGKIL
DELLQTCVDPEDCPVCEVAGRRFASGKKVTLNPSD
PEHCQICHCDVVNLTCEACQEPGGLVVPPTDAPVS
PTTLYVEDISEPPLHDF
SEQ ID NO: 4
amino acid sequence of full D′ defined
as ser764-asp864
SLSCRPPMVKLVCPADNLRAEGLECTKTCQNYDLE
CMSMGCVSGCLCPPGMVRHENRCVALERCPCFHQG
KEYAPGETVKIGCNTCVCRDRKWNCTDHVCD
SEQ ID NO: 5
amino acid sequence of full D3
defined as ala865-phe1270
ATCSTIGMAHYLTFDGLKYLFPGECQYVLVQDYCG
SNPGTFRILVGNKGCSHPSVKCKKRVTILVEGGEI
ELFDGEVNVKRPMKDETHFEVVESGRYIILLLGKA
LSVVWDRHLSISVVLKQTYQEKVCGLCGNFDGIQN
NDLTSSNLQVEEDPVDFGNSWKVSSQCADTRKVPL
DSSPATCHNNIMKQTMVDSSCRILTSDVFQDCNKL
VDPEPYLDVCIYDTCSCESIGDCACFCDTIAAYAH
VCAQHGKVVTWRTATLCPQSCEERNLRENGYECEW
RYNSCAPACQVTCQHPEPLACPVQCVEGCHAHCPP
GKILDELLQTCVDPEDCPVCEVAGRRFASGKKVTL
NPSDPEHcQICHCDVVNLTCEACQEPGGLVVPPTD
APVSPTTLYVEDISEPPLHDF
SEQ ID NO: 6
amino acid sequence of
CM110short764-863LWA
MIPARFAGVLLALALILPGTLCSLSCRPPMVKLVC
PADNLRAEGLECTKTCQNYDLECMSMGCVSGCLCP
PGMVRHENRCVALERCPCFHQGKEYAPGETVKIGC
NTCVCRDRKWNCTDHVCGGLTPRGVRLGGGSGGGS
GGGSEGGGSEGGGSEGGGSEGGGSEGGGSEGGGSG
GGSGSGGLTPRGVRLDAHKSEVAHRFKDLGEENFK
ALVLIAFAQYLQQCPFEDHVKLVNEVTEFAKTCVA
DESAENCDKSLHTLFGDKLCTVATLRETYGEMADC
CAKQEPERNECFLQHKDDNPNLPRLVRPEVDVMCT
AFHDNEETFLKKYLYEIARRHPYFYAPELLFFAKR
YKAAFTECCQAADKAACLLPKLDELRDEGKASSAK
QRLKCASLQKFGERAFKAWAVARLSQRFPKAEFAE
VSKLVTDLTKVHTECCHGDLLECADDRADLAKYIC
ENQDSISSKLKECCEKPLLEKSHCIAEVENDEMPA
DLPSLAADFVESKDVCKNYAEAKDVFLGMFLYEYA
RRHPDYSVVLLLRLAKTYETTLEKCCAAADPHECY
AKVFDEFKPLVEEPQNLIKQNCELFEQLGEYKFQN
ALLVRYTKKVPQVSTPTLVEVSRNLGKVGSKCCKH
PEAKRMPCAEDYLSVVLNQLCVLHEKTPVSDRVTK
CCTESLVNRRPCFSALEVDETYVPKEFNAETFTFH
ADICTLSEKERQIKKQTALVELVKHKPKATKEQLK
AVMDDFAAFVEKCCKADDKETCFAEEGKKLVAASQ
AALGL
SEQ ID NO: 7
amino acid sequence of linker in
CM110short764-863LWA
GGLTPRGVRLGGGSGGGSGGGSEGGGSEGGGSEGG
GSEGGGSEGGGSEGGGSGGGSGSGGLTPRGVRL
SEQ ID NO: 8
amino acid sequence of human serum
albumin residues 25-609
DAHKSEVAHRFKDLGEENFKALVLIAFAQYLQQCP
FEDHVKLVNEVTEFAKTCVADESAENCDKSLHTLF
GDKLCTVATLRETYGEMADCCAKQEPERNECFLQH
KDDNPNLPRLVRPEVDVMCTAFHDNEETFLKKYLY
EIARRHPYFYAPELLFFAKRYKAAFTECCQAADKA
ACLLPKLDELRDEGKASSAKQRLKCASLQKFGERA
FKAWAVARLSQRFPKAEFAEVSKLVTDLTKVHTEC
CHGDLLECADDRADLAKYICENQDSISSKLKECCE
KPLLEKSHCIAEVENDEMPADLPSLAADFVESKDV
CKNYAEAKDVFLGMFLYEYARRHPDYSVVLLLRLA
KTYETTLEKCCAAADPHECYAKVFDEFKPLVEEPQ
NLIKQNCELFEQLGEYKFQNALLVRYTKKVPQVST
PTLVEVSRNLGKVGSKCCKHPEAKRMPCAEDYLSV
VLNQLCVLHEKTPVSDRVTKCCTESLVNRRPCFSA
LEVDETYVPKEFNAETFTFHADICTLSEKERQIKK
QTALVELVKHKPKATKEQLKAVMDDFAAFVEKCCK
ADDKETCFAEEGKKLVAASQAALGL
SEQ ID NO: 9
Amino acid Sequence for a Thrombin
cleavage site
LTPRGVRL(SEQ ID NO: 9)
SEQ ID NO: 10
Amino acid Sequence for a factor Xa
cleavage site
I(E/D)GR
SEQ ID NO: 11:
amino acid sequence of CM115
MIPARFAGVLLALALILPGTLCSLSCRPPMVKLVC
PADNLRAEGLECTKTCQNYDLECMSMGCVSGCLCP
PGMVRHENRCVALERCPCFHQGKEYAPGETVKIGC
NTCVCRDRKWNCTDHVCDATCSTIGMAHYLTFDGL
KYLFPGECQYVLVQDYCGSNPGTFRILVGNKGCSH
PSVKCKKRVTILVEGGEIELFDGEVNVKRPMKDET
HFEVVESGRYIILLLGKALSVVWDRHLSISVVLKQ
TYQEKVCGLCGNFDGIQNNDLTSSNLQVEEDPVDF
GNSWKVSSQCADTRKVPLDSSPATCHNNIMKQTMV
DSSCRILTSDVFQDCNKLVDPEPYLDVCIYDTCSC
ESIGDCAAECEWFCDTIAAYAHVCAQHGKVVTWRT
ATLCPQSCEERNLRENGYEAEWRYNSCAPACQVTC
QHPEPLACPVQCVEGCHAHCPPGKILDELLQTCVD
PEDCPGGLTPRGVRLGGGSGGGSGGGSEGGGSEGG
GSEGGGSEGGGSEGGGSEGGGSGGGSGSGGLTPRG
VRLDAHKSEVAHRFKDLGEENFKALVLIAFAQYLQ
QCPFEDHVKLVNEVTEFAKTCVADESAENCDKSLH
TLFGDKLCTVATLRETYGEMADCCAKQEPERNECF
LQHKDDNPNLPRLVRPEVDVMCTAFHDNEETFLKK
YLYEIARRHPYFYAPELLFFAKRYKAAFTECCQAA
DKAACLLPKLDELRDEGKASSAKQRLKCASLQKFG
ERAFKAWAVARLSQRFPKAEFAEVSKLVTDLTKVH
TECCHGDLLECADDRADLAKYICENQDSISSKLKE
CCEKPLLEKSHCIAEVENDEMPADLPSLAADFVES
KDVCKNYAEAKDVFLGMFLYEYARRHPDYSVVLLL
RLAKTYETTLEKCCAAADPHECYAKVFDEFKPLVE
EPQNLIKQNCELFEQLGEYKFQNALLVRYTKKVPQ
VSTPTLVEVSRNLGKVGSKCCKHPEAKRMPCAEDY
LSVVLNQLCVLHEKTPVSDRVTKCCTESLVNRRPC
FSALEVDETYVPKEFNAETFTFHADICTLSEKERQ
IKKQTALVELVKHKPKATKEQLKAVMDDFAAFVEK
CCKADDKETCFAEEGKKLVAASQAALGL
MOLECULAR WEIGHT-122,714
PI-5.42

What is claimed is:

1. A coagulation factor complex comprising:
a. a factor VIII (FVIII) coagulation factor, or a FVIII coagulation factor fragment or variant thereof; and
b. a fusion protein comprising a D′D3short fragment of von Willebrand's factor, fused to full length albumin or, an albumin fragment or variant thereof, an immunoglobulin Fc domain, or an Fc fragment or variant thereof;

2. The coagulation factor complex of claim 1, wherein the fusion protein comprises a D′D3short fragment of von Willebrand's factor linked to a full length albumin or a fragment or variant thereof.

3. The coagulation factor complex of claim 2, wherein the albumin is full length albumin.

4. The coagulation factor complex of claim 1, further comprising a linker.

5. The coagulation factor complex of claim 4, wherein the linker is between the FVIII coagulation factor, or fragment or variant thereof, and the fusion protein.

6. The coagulation factor complex of claim 5, comprising click chemistry moieties in the linker.

7. The coagulation factor complex of claim 5, comprising PEG moieties in the linker.

8. The coagulation factor complex of claim 5, further comprising a cleavable amino acid moiety in the linker.

9. The coagulation factor complex of claim 8, wherein the amino acid linker is thrombin cleavable.

10. The coagulation factor complex of claim 8, wherein the amino acid linker is serine and glycine-rich.

11. The coagulation factor complex of claim 5, wherein the fusion protein comprises albumin and the linker between FVIII coagulation factor, or fragment or variant thereof, and the fusion protein occurs via cys34 of albumin.

12. The coagulation factor complex of claim 5, further comprising one or more cleavable amino acid linkers between the D′D3short fragment of von Willebrand's factor and the albumin, or albumin fragment or variant thereof or an immunoglobulin Fc domain, or an Fc fragment or variant thereof.

13. The coagulation factor complex of claim 5, comprising click chemistry moieties, PEG moieties, and a cleavable amino acid moiety in the linker.

14. The coagulation factor complex of claim 4, wherein the amino acid linker is selected from the group consisting of a (Gly₄Ser)ₙ linker, a (Gly₃Ser)ₙ linker, a (Gly₂Ser₄)ₙ linker, a (Gly₄Ser₂)ₙ linker, a (GlySer₅)ₙ linker, a (Gly)₆ linker, a (Gly)₈ linker, a GSAGSAAGSGEF linker, a KLTPRGVRLC linker, a GGSGGSLTPRGVLGGSWGGSC linker and GGLTPRGVRLGGGSGGGSGGGSEGGGSEGGGSEGGGSEGGGSEGGGSEGGGSGGGSGS GGLTPRGVRL linker, wherein n represents 1, 2, 3, 4, 5, 6, 7, 8, 9, 10, 11, 12, 13, 14, 15, 16, 17, 18, 19, 20, 25 repeats.

15. The coagulation factor complex of any of claims 1-14, wherein one, two, three or four of cys889, cyc898, cys1099, and cys1142 of the D′D3short, if present, have been replaced with an alanine.

16. The coagulation factor complex of any of claims 1-15, wherein the D′D3short fragment is selected from the group consisting of serine 764 through cysteine 1031, serine 764 through asparagine 864 (the full D′ domain), serine 764 through cysteine 863, leucine 765 through cysteine 863, leucine 765 through asparagine 864, serine 766 through cysteine 863, serine 766 through asparagine 864, serine 764 though arginine 1035, serine 764 though lysine 1036, serine 764 through serine 900, serine 764 through cysteine 1099, serine 764 through cysteine 1142, serine 764 though proline 1197 and serine 764 through proline 1240.

17. The coagulation factor complex of any of claims 1-15, wherein the D′D3short fragment is CM115.

18. The coagulation factor complex of claim 1, wherein the D′D3short fragment of the fusion protein is covalently fused to the full length albumin, albumin fragment or variant thereof, or immunoglobulin Fc domain, or Fc fragment or variant thereof.

19. The coagulation factor complex of claim 1, wherein the FVIII is full length factor VIII.

20. The coagulation factor complex of claim 1, wherein the FVIII fragment is B region deleted FVIII.

21. The coagulation factor complex of claim 1, wherein the FVIII coagulation factor and the fusion protein are coupled by click chemistry.

22. The coagulation factor complex of claim 1, wherein half-life of the coagulation factor complex is greater than at least 40 hours.

23. The coagulation factor complex of claim 1, wherein half-life of the coagulation factor complex is greater than at least 60 hours.

24. A fusion protein comprising a D′D3short fragment of von Willebrand's factor, fused to full length albumin, or an albumin fragment or a variant thereof, and/or an immunoglobulin Fc domain, or an Fc fragment or variant.

25. The fusion protein of claim 24, wherein the fusion protein comprises a D′D3short fragment of von Willebrand's factor fused to full length albumin or an albumin fragment or variant.

26. The fusion protein of claim 24, wherein the fusion is a covalent bond.

27. The fusion protein of claim 24, wherein the covalent bond is a peptide bond.

28. The fusion protein of claim 24, wherein the fusion protein is CM115.

29. The fusion protein of any one of claims 24-28, wherein the fusion protein is covalently bound to a FVIII coagulation factor via a linker comprising click chemistry moieties.

30. The fusion protein of claim 29, wherein the fusion protein is covalently bound to a FVIII coagulation factor via a linker further comprising a cleavable amino acid moiety.

31. The fusion protein of claim 29, wherein the covalent bond between FVIII and the fusion protein occurs via cys34 of albumin.

32. The fusion protein of claim 25 in a pharmaceutical carrier.

33. A kit comprising the composition of any one of claims 1-32.

34. A method of making a FVIII coagulation factor complex, the method comprising:
a. linking a D′D3short fragment of von Willebrand's factor to a full length albumin, an albumin fragment or variant thereof, or an immunoglobulin Fc domain, or a Fc fragment or variant thereof, to form a fusion protein;
b. linking the fusion protein of step a) to factor FVIII coagulation factor, or a fragment or variant thereof, wherein FVIII coagulation factor binds a receptor of D′D3short fragment, thereby forming a coagulation factor complex.

35. The method of claim 34, wherein the FVIII coagulation factor and the fusion protein are coupled by click chemistry.

36. The method of claim 34 or 35, wherein in the fusion protein the D′D3short fragment of von Willebrand's factor is linked by a covalent bond to a full length albumin, a albumin fragment or variant thereof, or an immunoglobulin Fc domain, or a Fc fragment or derivative thereof.

37. A method of treating a subject with a disease requiring FVIII coagulation factor infusion, comprising administering to the subject the coagulation factor complex of claim 1.

38. The method of claim 37, wherein the administration of the coagulation factor complex to the subject results in a blood level half-life of the coagulation factor complex which is greater than 40 hours.

39. The method of claim 37 or 38, wherein the coagulation factor complex is administered to the subject via injection.

40. The method of claim 39, wherein said coagulation factor complex is administered to the subject via subcutaneous injection.

41. The method of any one of claims 37-41, wherein the disease is hemophilia A.


SEQUENCE LISTING (11 sequences)

SEQ ID NO: 1 - DNA, 3459 bp, Artificial Sequence
  Synthetic Construct
atgatccccgccagattcgccggcgtgctgctggccctggccctgatcctgcctggcaccctgtgtagcctgagctgcagaccccccatggtcaagctcgtgtgccctgccgacaacctgcgggccgagggcctggaatgcaccaagacctgccagaactacgatctggaatgcatgagcatgggctgcgtgtccggctgcctgtgccctcctggaatggtccgacacgagaacagatgcgtggccctggaacggtgcccatgctttcatcaaggcaaagagtacgcccctggcgagacagtgaagatcggctgcaatacctgcgtgtgccgggaccggaagtggaactgcaccgaccacgtgtgcgacgccacctgtagcaccatcggcatggcccactacctgaccttcgacggcctgaagtacctgttccccggcgagtgccagtacgtgctggtgcaggactactgcggcagcaaccccggcaccttccggatcctcgtgggcaacaagggctgcagccaccccagcgtgaagtgcaagaagcgggtcaccatcctggtggaaggcggcgagatcgagctgttcgacggcgaagtgaacgtgaagcggcccatgaaggacgagacacacttcgaggtggtggaaagcggccggtacatcatcctgctgctgggcaaggccctgagcgtcgtgtgggaccggcacctgagcatcagcgtggtgctgaagcagacctaccaggaaaaagtctgcggcctctgcggcaacttcgacggcatccagaacaacgacctgaccagcagcaacctgcaggtcgaagaggaccccgtggacttcggcaacagctggaaggtgtccagccagtgcgccgacaccagaaaggtgcccctggacagcagccccgccacctgtcacaacaacatcatgaagcagacaatggtggacagctcctgccggatcctgaccagcgacgtgttccaggactgcaacaagctggtggaccccgagccctacctggacgtgtgcatctacgatacctgcagctgcgagagcatcggcgactgcgccgccttctgcgacacaatcgccgcctacgcccatgtgtgcgcccagcacggcaaggtggtcacctggcggaccgccaccctgtgcccacagagctgcgaggaacggaacctgcgcgagaacggctacgaggccgagtggcggtacaacagctgcgcccctgcctgccaggtcacatgccagcaccctgagcctctggcctgccccgtgcagtgcgtggaaggctgtcacgcccactgccctcccggcaagatcctggacgagctgctgcagacctgcgtggaccctgaggactgccctgtgtgcgaggtggccggcagaagattcgccagcggcaagaaagtgaccctgaaccccagcgaccccgagcactgccagatctgccactgcgacgtggtcaatctgacctgcgaggcctgtcaggaacctggcggcctggtcgtgcctcctggcggaagaggtggcggaggctctgggggaggttctggcggaggaagcgagggcggaggatctgagggtggcggctctgaaggtggcggaagcgaagggggaggctccgaaggcggtggatctgaaggggggggatctggcggcggatctggaagcggtggaagaggcgacgcccacaagtccgaggtggcccacagattcaaggacctgggcgaggaaaacttcaaggccctggtgctgatcgccttcgcccagtacctgcagcagtgccccttcgaggaccacgtgaagctggtcaacgaagtgaccgagttcgccaagacctgtgtggccgacgagagcgccgagaactgcgacaagagcctgcacaccctgttcggcgacaagctgtgcaccgtggccaccctgcgggaaacctacggcgagatggccgactgctgcgccaagcaggaacccgagcggaacgagtgcttcctgcagcacaaggacgacaaccccaacctgcccagactcgtgcggcccgaggtggacgtgatgtgcaccgccttccacgacaacgaggaaaccttcctgaagaagtacctctacgagatcgccagacggcacccctacttctacgcccccgagctgctgttcttcgccaagcggtacaaggccgccttcaccgagtgctgccaggccgccgataaggccgcctgcctgctgcccaagctggatgagctgagggacgagggcaaggccagctccgccaagcagagactgaagtgcgccagcctgcagaagttcggcgagcgggcctttaaggcctgggccgtggcccggctgagccagagattccccaaggccgagtttgccgaggtgtccaagctggtcaccgatctgaccaaggtgcacaccgagtgttgtcacggcgacctgctggaatgcgccgacgacagagccgacctggccaagtacatctgcgagaaccaggacagcatcagctccaagctgaaagagtgctgcgagaagcccctgctggaaaagagccactgtatcgccgaggtggaaaacgacgagatgcccgccgacctgcccagcctggccgccgacttcgtggaaagcaaggatgtgtgcaagaactacgccgaggccaaggacgtgttcctgggcatgttcctgtacgagtacgcccgcagacaccccgactactccgtggtgctgctgctgcggctggccaagacctacgagacaaccctggaaaagtgctgcgccgctgccgacccccacgagtgctacgccaaggtgttcgacgagttcaagcctctggtggaagaaccccagaacctgatcaagcagaactgcgagctgtttgagcagctgggcgagtacaagttccagaacgccctgctcgtgcggtacaccaagaaagtgcctcaggtgtccacccccacactggtggaagtgtcccggaacctgggcaaagtgggctccaagtgctgcaagcacccagaggccaagcggatgccctgcgccgaggactacctgagcgtggtcctgaaccagctgtgcgtgctgcacgagaaaacccccgtgtccgacagagtgaccaagtgctgtaccgagagcctggtcaacagacggccctgcttcagcgccctggaagtggacgagacatacgtgcccaaagagttcaacgccgagacattcaccttccacgccgacatctgcaccctgtccgagaaagagcggcagatcaagaagcagaccgccctggtcgagctggtcaagcacaagcccaaggccaccaaagaacagctgaaggccgtgatggacgacttcgccgccttcgtcgagaagtgttgcaaggccgacgacaaagagacatgcttcgccgaagagggcaagaaactggtggccgcctctcaggccgccctgggcctgcaccaccaccaccaccac

SEQ ID NO: 2 - DNA, 3378 bp, Artificial Sequence
  Synthetic Construct
atgagcctgagctgcagaccccccatggtcaagctcgtgtgccctgccgacaacctgcgggccgagggcctggaatgcaccaagacctgccagaactacgatctggaatgcatgagcatgggctgcgtgtccggctgcctgtgccctcctggaatggtccgacacgagaacagatgcgtggccctggaacggtgcccatgctttcatcaaggcaaagagtacgcccctggcgagacagtgaagatcggctgcaatacctgcgtgtgccgggaccggaagtggaactgcaccgaccacgtgtgcgacgccacctgtagcaccatcggcatggcccactacctgaccttcgacggcctgaagtacctgttccccggcgagtgccagtacgtgctggtgcaggactactgcggcagcaaccccggcaccttccggatcctcgtgggcaacaagggctgcagccaccccagcgtgaagtgcaagaagcgggtcaccatcctggtggaaggcggcgagatcgagctgttcgacggcgaagtgaacgtgaagcggcccatgaaggacgagacacacttcgaggtggtggaaagcggccggtacatcatcctgctgctgggcaaggccctgagcgtcgtgtgggaccggcacctgagcatcagcgtggtgctgaagcagacctaccaggaaaaagtctgcggcctctgcggcaacttcgacggcatccagaacaacgacctgaccagcagcaacctgcaggtcgaagaggaccccgtggacttcggcaacagctggaaggtgtccagccagtgcgccgacaccagaaaggtgcccctggacagcagccccgccacctgtcacaacaacatcatgaagcagacaatggtggacagctcctgccggatcctgaccagcgacgtgttccaggactgcaacaagctggtggaccccgagccctacctggacgtgtgcatctacgatacctgcagctgcgagagcatcggcgactgcgccgccttctgcgacacaatcgccgcctacgcccatgtgtgcgcccagcacggcaaggtggtcacctggcggaccgccaccctgtgcccacagagctgcgaggaacggaacctgcgcgagaacggctacgaggccgagtggcggtacaacagctgcgcccctgcctgccaggtcacatgccagcaccctgagcctctggcctgccccgtgcagtgcgtggaaggctgtcacgcccactgccctcccggcaagatcctggacgagctgctgcagacctgcgtggaccctgaggactgccctgtgtgcgaggtggccggcagaagattcgccagcggcaagaaagtgaccctgaaccccagcgaccccgagcactgccagatctgccactgcgacgtggtcaatctgacctgcgaggcctgtcaggaacctggcggcctggtcgtgcctcctggcggaagaggtggcggaggctctgggggaggttctggcggaggaagcgagggcggaggatctgagggtggcggctctgaaggtggcggaagcgaagggggaggctccgaaggcggtggatctgaaggggggggatctggcggcggatctggaagcggtggaagaggcgacgcccacaagtccgaggtggcccacagattcaaggacctgggcgaggaaaacttcaaggccctggtgctgatcgccttcgcccagtacctgcagcagtgccccttcgaggaccacgtgaagctggtcaacgaagtgaccgagttcgccaagacctgtgtggccgacgagagcgccgagaactgcgacaagagcctgcacaccctgttcggcgacaagctgtgcaccgtggccaccctgcgggaaacctacggcgagatggccgactgctgcgccaagcaggaacccgagcggaacgagtgcttcctgcagcacaaggacgacaaccccaacctgcccagactcgtgcggcccgaggtggacgtgatgtgcaccgccttccacgacaacgaggaaaccttcctgaagaagtacctctacgagatcgccagacggcacccctacttctacgcccccgagctgctgttcttcgccaagcggtacaaggccgccttcaccgagtgctgccaggccgccgataaggccgcctgcctgctgcccaagctggatgagctgagggacgagggcaaggccagctccgccaagcagagactgaagtgcgccagcctgcagaagttcggcgagcgggcctttaaggcctgggccgtggcccggctgagccagagattccccaaggccgagtttgccgaggtgtccaagctggtcaccgatctgaccaaggtgcacaccgagtgttgtcacggcgacctgctggaatgcgccgacgacagagccgacctggccaagtacatctgcgagaaccaggacagcatcagctccaagctgaaagagtgctgcgagaagcccctgctggaaaagagccactgtatcgccgaggtggaaaacgacgagatgcccgccgacctgcccagcctggccgccgacttcgtggaaagcaaggatgtgtgcaagaactacgccgaggccaaggacgtgttcctgggcatgttcctgtacgagtacgcccgcagacaccccgactactccgtggtgctgctgctgcggctggccaagacctacgagacaaccctggaaaagtgctgcgccgctgccgacccccacgagtgctacgccaaggtgttcgacgagttcaagcctctggtggaagaaccccagaacctgatcaagcagaactgcgagctgtttgagcagctgggcgagtacaagttccagaacgccctgctcgtgcggtacaccaagaaagtgcctcaggtgtccacccccacactggtggaagtgtcccggaacctgggcaaagtgggctccaagtgctgcaagcacccagaggccaagcggatgccctgcgccgaggactacctgagcgtggtcctgaaccagctgtgcgtgctgcacgagaaaacccccgtgtccgacagagtgaccaagtgctgtaccgagagcctggtcaacagacggccctgcttcagcgccctggaagtggacgagacatacgtgcccaaagagttcaacgccgagacattcaccttccacgccgacatctgcaccctgtccgagaaagagcggcagatcaagaagcagaccgccctggtcgagctggtcaagcacaagcccaaggccaccaaagaacagctgaaggccgtgatggacgacttcgccgccttcgtcgagaagtgttgcaaggccgacgacaaagagacatgcttcgccgaagagggcaagaaactggtggccgcctctcaggccgccctgggcctg

SEQ ID NO: 3 - protein, 507 aa, Artificial Sequence
  Synthetic Construct
SLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCAAFCDTIAAYAHVCAQHGKVVTWRTATLCPQSCEERNLRENGYEAEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPPTDAPVSPTTLYVEDISEPPLHDF

SEQ ID NO: 4 - protein, 101 aa, Artificial Sequence
  Synthetic Construct
SLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCD

SEQ ID NO: 5 - protein, 406 aa, Artificial Sequence
  Synthetic Construct
ATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPPTDAPVSPTTLYVEDISEPPLHDF

SEQ ID NO: 6 - protein, 775 aa, Artificial Sequence
  Synthetic Construct
MIPARFAGVLLALALILPGTLCSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCGGLTPRGVRLGGGSGGGSGGGSEGGGSEGGGSEGGGSEGGGSEGGGSEGGGSGGGSGSGGLTPRGVRLDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 7 - protein, 68 aa, Artificial Sequence
  Synthetic Construct
GGLTPRGVRLGGGSGGGSGGGSEGGGSEGGGSEGGGSEGGGSEGGGSEGGGSGGGSGSGGLTPRGVRL

SEQ ID NO: 8 - protein, 585 aa, Artificial Sequence [sequence omitted]

SEQ ID NO: 9 - protein, 8 aa, Artificial Sequence
  Synthetic Construct
LTPRGVRL

SEQ ID NO: 10 - protein, 4 aa, Artificial Sequence
  Synthetic Construct
  misc_feature (2)..(2): Xaa can be any naturally occurring amino acid
IXGR

SEQ ID NO: 11 - protein, 1113 aa, Artificial Sequence
  Synthetic Construct
MIPARFAGVLLALALILPGTLCSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCAAECEWFCDTIAAYAHVCAQHGKVVTWRTATLCPQSCEERNLRENGYEAEWRYNSCAPACQVTCQHPEPLACPVQCVEGCHAHCPPGKILDELLQTCVDPEDCPGGLTPRGVRLGGGSGGGSGGGSEGGGSEGGGSEGGGSEGGGSEGGGSEGGGSGGGSGSGGLTPRGVRLDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL